(19) United States
(12) Patent Application Publication
(10) Pub. No.: US 2024/0352532 A1
(43) Pub. Date: Oct. 24, 2024

(54) METHODS FOR CATEGORIZING DISEASE OUTCOMES

(71) Applicants: Akira YOKOI, Tokyo (JP); Kosuke YOSHIDA, Tokyo (JP); Craif Inc., Tokyo (JP); NATIONAL CANCER CENTER, Tokyo (JP)

(72) Inventors: Akira YOKOI, Tokyo (JP); Kosuke YOSHIDA, Tokyo (JP); Hiroaki KAJIYAMA, Tokyo (JP); Tomoyasu KATO, Tokyo (JP); Yusuke YAMAMOTO, Tokyo (JP)

(21) Appl. No.: 18/618,157

(22) Filed: Mar. 27, 2024

Related U.S. Application Data
(63) Continuation of application No. PCT/JP2022/037272, filed on Oct. 5, 2022.
(60) Provisional application No. 63/318,105, filed on Mar. 9, 2022.
(60) Provisional application No. 63/357,964, filed on Jul. 1, 2022.

(51) Int. Cl. C12Q 1/6886; A61B 5/00; C12Q 1/6874

(52) U.S. Cl. CPC C12Q 1/6886; C12Q 1/6874; A61B 5/4325; C12Q 2600/178

(57) ABSTRACT

Disclosed herein are methods for predicting the clinical or disease outcome of a subject with a disease. The methods for generating an index to predict the disease outcome are also provided. The methods comprise procedures for measuring miRNA levels in a cell-free sample of a subject. The compositions for practicing the same are further provided.

### CROSS-REFERENCE

This application is continuation of International Application No. PCT/JP2022/037272 filed Oct. 5, 2022, which claims priority to U.S. Patent Application No. 63/318,105, filed Mar. 9, 2022, and U.S. Patent Application No. 63/357,964, filed Jul. 1, 2022, each of which is incorporated by reference herein in its entirety.

### TECHNICAL FIELD

Cancer is one of the leading causes of death, and its prevalence is escalating dramatically. Accurate cancer outcome prediction and/or cancer categorization is a beneficial aspect of cancer care, and various diagnostics such as imaging, biopsies, and various laboratory-based tests are used to accomplish that goal. However, current methods lack sufficient sensitivity and/or accuracy due at least in part of the complex interactions between types/subtypes of different cancers and various extrinsic (e.g., environmental factors) and/or intrinsic factors (e.g., genotypes) of the subjects. The current methods are insufficient and ineffective to predict disease outcome, hindering the deployment of the effective treatments. Patients with more severe disease outcomes may require treatments that entail additional resources (e.g., dosage or frequency of the treatments) and vice versa. Additionally, current methods for cancer predictions may require extensively invasive procedures, preventing population-wide application of the methods and characterization of the cancers, which in turn can contribute to ineffective prediction of the cancer outcome prediction and/or cancer categorization. In some cases, current methods may not prevent early-stage disease detection. In other cases, patients can only be diagnosed at an advanced stage with extensive metastasis, contributing to a high lethality of cancer.

### SUMMARY OF INVENTION

Proper cancer outcome prediction and/or cancer categorization can lead to effective cancer treatments. Provided herein are methods, compositions, or kits for generating an index for predicting disease outcome in a subject. The disease may comprise a cancer. The index may help accurately, effectively, and/or efficiently predict the disease outcome of a subject such that an appropriate treatment option can be prescribed for the subject. In some cases, the methods may comprise stratifying a population of subjects with a similar type or subtype of cancer, thereby minimizing the differences of the cancer types/subtypes that may confound disease outcome prediction. In some cases, the accurate prediction of the disease outcome may facilitate the prescription of effective treatments to a subject according to the disease condition. In some cases, the accurate prediction of the disease outcome may reduce the morality of the disease improve the quality of the subject's remaining life-span. In some aspects, miRNAs in cell-free sample can be used to accurately, effectively, and/or efficiently predict the disease outcome. The miRNA may remain stable in the cell-free sample if they are encompassed by extracellular vesicles. These methods and/or compositions can allow for accurate prediction of disease outcomes using the cell-free samples or samples obtained from non-invasive samplings. Such methods may facilitate population-wide cancer outcome prediction and/or cancer categorization, which in turn can lead to increasingly accurate cancer outcome prediction and/or cancer categorization. In some cases, the methods provided herein can allow disease outcome prediction using more than one biomarker, which may provide increased accuracy compared to the methods using only one biomarker. These methods can also allow for identification of biomarkers specific for disease outcome prediction, disease categorization, or a combination thereof. In some cases, the methods may also allow for early-detection of the disease,

Provided herein, are methods. In an aspect, a method may comprise: (a) obtaining an index derived from at least two micro-ribonucleic acids (miRNAs) of a subject having an ovarian cancer; and (b) determining an outcome of the ovarian cancer in the subject, wherein the at least two miRNAs are obtained from a cell-free sample of the subject.

In some embodiments, the ovarian cancer comprises a type I ovarian cancer, a type II ovarian cancer, or a combination thereof. In some embodiments, the ovarian cancer comprises the type I ovarian cancer. In some embodiments, the type I ovarian cancer comprises endometrioid carcinoma, ovarian clear cell carcinoma, mucinous carcinoma, low-grade serous carcinoma, or a combination thereof. In some embodiments, the type I ovarian cancer comprises the endometrioid carcinoma. In some embodiments, the type I ovarian cancer comprises the ovarian clear cell carcinoma. In some embodiments, the type I ovarian cancer comprises the mucinous carcinoma. In some embodiments, the type I ovarian cancer comprises the low-grade serous carcinoma. In some embodiments, the ovarian cancer comprises the type II ovarian cancer. In some embodiments, the type II ovarian cancer comprises high-grade serous ovarian carcinoma. In some embodiments, the ovarian cancer comprises epithelial ovarian carcinoma, germ cell tumor, stromal cell tumor, or a combination thereof. In some embodiments, the ovarian cancer comprises the epithelial ovarian carcinoma. In some embodiments, the ovarian cancer comprises the germ cell tumor. In some embodiments, the ovarian cancer comprises the stromal cell tumor.

In some embodiments, the at least two miRNAs comprise miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, miR-6805-5p, or a combination thereof. In some embodiments, the at least two miRNAs comprise miR-187-5p, miR-6870-5p, miR-1908-5p, miR-6727-5p, or a combination thereof. In some embodiments, the at least two miRNAs comprise miR-187-5p. In some embodiments, the at least two miRNAs comprise miR-6870-5p. In some embodiments, the at least two miRNAs comprise miR-1908-5p. In some embodiments, the at least two miRNAs comprise miR-6727-5p. In some embodiments, the at least two miRNAs comprise miR-187-5p and miR-6870-5p. In some embodiments, the at least two miRNAs comprise miR-187-5p, miR-6870-5p, and miR-1908-5p. In some embodiments, the at least two miRNAs comprise miR-187-5p, miR-6870-5p, and miR-6727-5p. In some embodiments, the at least two miRNAs comprise miR-187-5p, miR-6870-5p, miR-1908-5p, and miR-6727-5p. In some embodiments, the at least two miRNAs comprise miR-150-3p, miR-3195, miR-7704, or a combination thereof. In some embodiments, the at least two miRNAs comprise miR-150-3p. In some embodiments, the at least two miRNAs comprise miR-3195. In some embodiments, the at least two miRNAs comprise miR-7704. In some embodiments, the at least two miRNAs comprise miR-150-3p, miR-3195 and miR-7704.

In some embodiments, the index is derived from levels of the at least two miRNAs. In some embodiments, the levels of the at least two miRNAs comprise expression levels of the at least two miRNAs or derivatives of the expression levels of the at least two miRNAs. In some embodiments, the index is obtained by processing the levels of the at least two miRNAs of at least two subjects having the ovarian cancer. In some embodiments, the index is obtained by processing the levels of the at least two miRNAs of at least 10 subjects having the ovarian cancer. In some embodiments, the index is obtained by processing the levels of the at least at least two miRNAs of at least 50 subjects having the ovarian cancer. In some embodiments, the index is obtained by processing the levels of the at least at least two miRNAs of at least 100 subjects having the ovarian cancer. In some embodiments, the index comprises a formula comprising the levels of the at least two miRNAs. In some embodiments, the formula comprises: (a) 0.218×(a level of miR-187-5p)+0.280×(a level of miR-6870-5p); or (b) 0.148×(said level of miR-187-5p)+0.273×(said level of miR-6870-5p)+0.186×(a level of miR-1908-5p); or (c) 0.034×(said level of miR-187-5p)+0.236×(said level of miR-6870-5p)+0.504×(a level of miR-6727-5p)+0.048×(said level of miR-1908-5p); or (d) 0.031×(said level of miR-187-5p)+0.231 (said level of miR-6870-5p)+0.351×(said level of miR-6727-5p).

In some embodiments, the formula comprises: (a) 0.463×(a level of miR-150-3p)+1.323×(a level of miR-3195)+0.636×(a level of miR-7704); or (b) 0.399×(said level of miR-150-3p)+1.426×(said level of miR-3195)+0.480×(said level of miR-7704).

In some embodiments, the index is obtained by processing the levels of the at least two miRNAs using an algorithm. In some embodiments, the algorithm comprises a statistical model. In some embodiments, the statistical model comprises a linear regression model. In some embodiments, the linear regression model comprises a Cox model. In some embodiments, the Cox model comprises a univariate Cox model or a multivariate Cox model. In some embodiments, the Cox model comprises the univariate Cox model. In some embodiments, the Cox model comprises the multivariate Cox model. In some embodiments, the levels of the at least two miRNAs are determined by a microarray, a sequencing reaction, a probe hybridization, a polymerase chain reaction (PCR), or a combination thereof. In some embodiments, the levels of the at least two miRNAs are determined by the microarray.

In some embodiments, the ovarian cancer is a stage I ovarian cancer, a stage II ovarian cancer, a stage III ovarian cancer, or a stage IV ovarian cancer. In some embodiments, the stage I ovarian cancer comprises a stage IA ovarian cancer or a stage IB ovarian cancer. In some embodiments, the stage II ovarian cancer comprises a stage IIA ovarian cancer or a stage IIB ovarian cancer. In some embodiments, the stage III ovarian cancer comprises a stage IIIA ovarian cancer, a stage IIIB ovarian cancer, or a stage IIIC ovarian cancer. In some embodiments, the outcome comprises an amount of time that the ovarian cancer has not progressed to a succeeding stage, during or subsequent to when the subject has received a treatment for the ovarian cancer. In some embodiments, the outcome comprises an amount of time that the subject is alive, subsequent to the subject has been determined to have the ovarian cancer or the subject has received a treatment for the ovarian cancer. In some embodiments, the outcome comprises progress-free survival (PFS) or overall survival (OS). In some embodiments, the outcome comprises the PFS. In some embodiments, the comprises the OS.

In some embodiments, the cell-free sample of the subject comprises a bodily fluid of the subject. In some embodiments, the bodily fluid comprises serum, urine, sweat, plasma, tears, semen, vaginal fluid, amniotic fluid, milk, or a combination thereof. In some embodiments, the bodily fluid sample comprises the serum. In some embodiments, the at least two miRNAs are derived from extracellular vesicles of the subject.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material . . .

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” or “FIGs.” herein), of which:

FIG. 1 depicts a schematic of patients and microRNAs (miRNAs) selection.

FIG. 2 depicts Kaplan-Meier curves showing overall survival (OS) of patients with high-grade serous ovarian carcinoma stratified according to serum miRNA levels. Patients were stratified according to the median level of each miRNA.

FIG. 3 depicts the expression levels of miR-187-5p, miR-6870-5p, miR-1908-5p, miR-6727-5p, and miR-6850-5p in the serum of subjects with high-grade serous ovarian cancer (HGSOC) relative to the healthy subjects.

FIG. 4A depicts Kaplan-Meier curves showing overall survival (OS) stratified with the median of an example index. n depicts the number of subjects.

FIG. 4B depicts Kaplan-Meier curves showing OS stratified with the median of another example index. n depicts the number of subjects.

FIG. 5A depicts Kaplan-Meier curves showing progression-free survival (PFS) stratified with the median of an example index. n depicts the number of subjects.

FIG. 5B depicts Kaplan-Meier curves showing PFS stratified with the median of another example index. n depicts the number of subjects.

FIG. 6A depicts Kaplan-Meier curves showing OS stratified with the high, intermediate, and low example indices. n depicts the number of subjects. P-value (p) for various comparisons between index groups are shown.

FIG. 6B depicts Kaplan-Meier curves showing PFS stratified with the high, intermediate, and low example indices. n depicts the number of subjects. P-value (p) for various comparisons between index groups are shown.

FIG. 6C depicts a correlation plot between the example index for OS of FIG. 6A and the example index for PFS of FIG. 6B. The correlation coefficient square (R²) and P-value (p) are shown.

FIG. 7A depicts relative expression levels of miR-187-5p, miR-1908-5p, and miR-6870-5p transfected (mimics) in A2780 and SK-OV-3 cell lines. NC depicts negative controls.

FIG. 7B depicts proliferation assay of cells transfected with miRNAs in FIG. 7A.

FIG. 7C depicts proliferation assay of cells transfected with miRNAs in FIG. 7A in the presences of various drugs.

FIG. 8 depicts a schematic of patient selection.

FIG. 9A depicts Hazard ratios (HRs) and 95% confidence intervals (CIs) for overall survival (OS) calculated using the levels of miRNAs as a continuous variable. The heatmap shows the expression of miRNAs derived from ovarian clear cell carcinoma tissue.

FIG. 9B depicts Hazard ratios (HRs) and 95% confidence intervals (CIs) for progression-free survival (PFS) calculated using the levels of miRNAs as a continuous variable. The heatmap shows the expression of miRNAs derived from ovarian clear cell carcinoma tissue.

FIG. 10 depicts Kaplan-Meier curves showing OS and PFS of patients with ovarian clear cell carcinoma stratified according to serum microRNA levels. Patients were stratified according to the median level of each microRNA.

FIG. 11 depicts a schematic for miRNA candidates selection using univariate and multivariate Cox regression analysis of patients with clear cell carcinoma according to serum microRNA levels and selected by the expression of miRNAs derived from ovarian clear cell carcinoma (OCCC) tissue.

FIG. 12A depicts Kaplan-Meier curves showing OS stratified with the median of the index-OS. Patients were stratified according to the median level of each microRNA.

FIG. 12B depicts Kaplan-Meier curves showing PFS stratified with the median of the index-OS. Patients were stratified according to the median level of each microRNA.

FIG. 13A depicts serum levels of miRNAs stratified with the median of an example index for OS and an example index for PFS.

FIG. 13B depicts a correlation plot between the example index for OS and the example index for PFS of FIG. 13A.

### DETAILED DESCRIPTION

### Definitions

The term “cancer” as used herein, refers to or describes the physiological condition in mammals that comprises unregulated cell growth. Cancer may also comprise a cell or tissue that comprises unregulated cell growth.

The terms “determining,” “measuring,” “evaluating,” “assessing,” “assaying,” and “analyzing” as used herein, refer to forms of measurement. The terms include determining if an element is present or not (for example, detection). These terms can include quantitative, qualitative or quantitative and qualitative determinations. These terms can include relative or absolute determinations.

The terms “subject” as used herein refers to a biological entity. The subject can be a mammal. The mammal can be a human.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “at most,” “no more than,” “less than,” or “less than or equal to” precedes the first numerical value in a series of two or more numerical values, the term “no more than” or “less than” applies to each one of the numerical values in that series of numerical values.

As used herein, the singular forms “a”, “an”, and “the” include plural references unless the context clearly dictates otherwise.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

### Methods

In some instances, the methods described herein may comprise generating an index. The index may be used to predict the clinical outcome or disease outcome of a subject with a disease in a subject. Provided herein are the compositions or kits to practice the methods described herein.

A method may comprise obtaining an index derived from a micro-ribonucleic acid (miRNA). A method may comprise obtaining an index derived from a micro-ribonucleic acid (miRNA) of a subject. The subject may have a cancer. A method may comprise determining an outcome of a subject with a cancer. A method may comprise obtaining an index derived from at least two micro-ribonucleic acids (miRNAs) of a subject. A method may comprise determining an outcome of an ovarian cancer in a subject, wherein at least two miRNAs are obtained from a cell-free sample of a subject. A method may comprise: (a) obtaining an index derived from at least two micro-ribonucleic acids (miRNAs) of a subject having an ovarian cancer; and (b) determining an outcome of the ovarian cancer in said subject, wherein the at least two miRNAs are obtained from a cell-free sample of the subject. A method may comprise the steps of: (a) obtaining an index derived from at least two micro-ribonucleic acids (miRNAs) of a subject having an ovarian cancer; and (b) determining an outcome of the ovarian cancer in the subject, wherein the at least two miRNAs are obtained from a cell-free sample of said subject.

### Cancer

In some instances, the methods described herein may predict the disease outcome of a subject with a cancer. The methods may predict the disease outcome of a subject with an ovarian cancer. In some instances, a method may predict the clinical outcome or disease outcome of a cancer type in a subject. In some instances, a method may predict the clinical outcome or disease outcome of a cancer subtype in a subject. In some instances, a method may predict the clinical outcome or disease outcome of an ovarian cancer type in a subject. In some instances, a method may predict the clinical outcome or disease outcome of an ovarian cancer subtype in a subject.

In some instances, there can be two types of ovarian cancer. In some instances, the two types of ovarian cancer are Type I ovarian carcinomas and Type II ovarian carcinomas. In some instances, the Type I ovarian carcinomas can be slow-growing, indolent neoplasms arising from a precursor lesion in the ovaries. In some instances, Type I ovarian carcinomas can include endometroid carcinoma, clear cell carcinoma, mucinous carcinoma, or low-grade serous carcinoma. In some instances, the Type II carcinomas can be clinically aggressive neoplasms that can develop de novo from serous tubal intraepithelial carcinomas (STIC) and/or ovarian surface epithelium. In some instances, Type II carcinomas can include high-grade serous carcinoma (HGSOC).

In some instances, an ovarian cancer type may comprise epithelial ovarian carcinomas, germ cell tumors, stromal cell tumors, or a combination thereof. In some instance, epithelial ovarian carcinomas can involve cancer cells covering the outer surface of the ovary. These cancer cells can spread to the lining and organs of the pelvis and abdomen and then to other parts of the body. In some instances, germ cell tumors can begin in the reproductive cells (e.g., the eggs) of the ovaries. In some instances, stromal cell tumors can form in the tissues that support the ovaries. In some instances, a method may predict the disease outcome of a subject with two of epithelial ovarian carcinomas, germ cell tumors, stromal cell tumors. In some instances, a method may predict the disease outcome of a subject with an epithelial ovarian carcinomas, germ cell tumors, stromal cell tumors. In some instances, a method may predict the disease outcome of a subject with the epithelial ovarian carcinomas. In some instances, a method may predict the disease outcome of a subject with the germ cell tumors. In some instances, a method may predict the disease outcome of a subject with the stromal cell tumors.

The subjects may have an endometrioid ovarian cancer, a mucinous ovarian cancer, a serous ovarian cancer, a clear cell ovarian cancer, or a combination thereof. The subjects may have endometrioid or mucinous ovarian cancer. The subjects may have endometrioid or serous ovarian cancer. The subjects may have mucinous or serous ovarian cancer. In some instances, a method may predict the disease outcome of a subject with at least two of an endometrioid ovarian cancer, a mucinous ovarian cancer, a serous ovarian cancer, and a clear cell ovarian cancer. In some instances, a method may predict the disease outcome of a subject with at least three of an endometrioid ovarian cancer, a mucinous ovarian cancer, a serous ovarian cancer, and a clear cell ovarian cancer. In some instances, a method may predict the disease outcome of a subject with an endometrioid ovarian cancer, a mucinous ovarian cancer, a serous ovarian cancer, and a clear cell ovarian cancer.

In some instances, a method may predict the disease outcome of a subject with type I or type II ovarian carcinoma. In some instances, a method may predict the disease outcome of a subject with type I and type II ovarian carcinoma. In some instances, a method may predict the disease outcome of a subject with type I ovarian carcinoma. In some instances, a method may predict the disease outcome of a subject with type II ovarian carcinoma. In some cases, a method may predict the disease outcome of a subject with endometroid carcinoma. In some cases, a method may predict the disease outcome of a subject with clear cell carcinoma. In some cases, a method may predict the disease outcome of a subject with mucinous carcinoma. In some cases, a method may predict the disease outcome of a subject with low-grade serous carcinoma. In some cases, a method may predict the disease outcome of a subject with HGSOC. In some cases, a method may predict the disease outcome of a subject with endometroid carcinoma, clear cell carcinoma, mucinous carcinoma, low-grade serous carcinoma, HGSOC, or a combination thereof. In some cases, a method may predict the disease outcome of a subject with at least two of endometroid carcinoma, clear cell carcinoma, mucinous carcinoma, low-grade serous carcinoma, and HGSOC. In some cases, a method may predict the disease outcome of a subject with at least three of endometroid carcinoma, clear cell carcinoma, mucinous carcinoma, low-grade serous carcinoma, and HGSOC. In some cases, a method may predict the disease outcome of a subject with at least four of endometroid carcinoma, clear cell carcinoma, mucinous carcinoma, low-grade serous carcinoma, and HGSOC. In some cases, a method may predict the disease outcome of a subject with endometroid carcinoma, clear cell carcinoma, mucinous carcinoma, low-grade serous carcinoma, and HGSOC.

In some cases, the cancer may have a cancer stage. The cancer may comprise a stage I cancer, a stage II cancer, a stage III cancer, a stage IV cancer, or a combination thereof. A cancer may also comprise a stage 0 cancer, a stage I cancer, a stage II cancer, a stage III cancer, or a stage IV cancer. The cancer may comprise a stage 0 cancer. The cancer may comprise a stage I cancer. The cancer may comprise a stage II cancer. The cancer may comprise a stage III cancer. The cancer may comprise a stage IV cancer.

A subject with a stage 0 cancer may not have the cancer but is at risk of developing a cancer. For example, the subject may have neoplastic cells that have the potential to develop into a cancer. A stage I cancer may comprise a small cancer. A stage I cancer may be localized to one area, tissue, or organ. A stage I cancer may be an early-stage cancer. A stage I cancer may not have grown deeply into a tissue adjacent its origin. A stage I cancer may not have grown into a lymph node. In some instances, a stage I cancer may comprise a stage IA cancer or a stage IB cancer. In some instances, a stage I cancer may comprise a stage IA cancer. In some instances, a stage I cancer may comprise a stage IB cancer. A stage IA cancer may comprise stage I cancer with a tumor with at most about 2 centimeters (cm) in cross section. A stage IB cancer may comprise stage I cancer with a tumor with at least about 2 cm in cross section. A stage IB cancer may comprise stage I cancer with a tumor with at most about 4 cm in cross section. A stage IB cancer may comprise stage I cancer with a tumor with about 2 to 4 cm in cross section.

In some instances, a stage II or III cancer may comprise a cancer that has grown into a tissue adjacent its origin or lymph node. In some instances, a stage II cancer may comprise a cancer that has not grown into a lymph node. A stage II cancer is larger in size, volume, or weight than a stage I cancer. In some instances, a stage II cancer may comprise a stage IIA cancer or a stage IIB cancer. In some instances, a stage II cancer may comprise a stage IIA cancer. In some instances, a stage II cancer may comprise a stage IIB cancer. A stage IIA cancer may comprise stage II cancer with a tumor with at least about 4 cm in cross section and has not spread to a lymph node. A stage IIB cancer may comprise stage II cancer that has spread to at most about 3 lymph nodes. A stage IIB cancer may comprise stage II cancer with at most about 2 cm in cross section and has spread to at most about 3 lymph nodes. A stage IIB cancer may comprise stage II cancer from about 2 to 4 in cross section and has spread to at most about 3 lymph nodes. A stage IIB cancer may comprise stage II cancer with at least about 4 in cross section and has spread to at most about 3 lymph nodes.

A stage III cancer is larger in size, volume, or weight than a stage II cancer. A stage III cancer may have a deeper penetration into a tissue than a stage II cancer does. In some instances, a stage III cancer may have spread to at least 4 lymph nodes. In some instances, a stage III cancer may comprise a stage IIIA cancer, a stage IIIB cancer, or a stage IIIC cancer. In some instances, a stage III cancer may comprise a stage IIIA cancer. In some instances, a stage III cancer may comprise a stage IIIB cancer. In some instances, a stage III cancer may comprise a stage IIIC cancer. A stage IIIA cancer may comprise stage III cancer that is at most about 2 cm in cross section and has spread to at least about 4 lymph nodes. A stage IIIB cancer may comprise stage III cancer that is about 2 to 4 cm in cross section and has spread to at least about 4 lymph nodes. A stage IIIB cancer may comprise stage III cancer that is at least about 4 cm in cross section and has spread to at least about 4 lymph nodes.

In some instances, a stage IV cancer may comprise a cancer that has spread to other organs or parts of a subject, relative to the part/tissue the cancer originates. In some cases, a stage IV cancer may comprise an advanced or metastatic cancer.

In some cases, a method may predict the disease outcome of a subject with a cancer with a cancer stage. In some cases, a method may predict the disease outcome of a subject with a stage 0 cancer, a stage I cancer, a stage II cancer, a stage III cancer, or a stage IV cancer. In some cases, a method may predict the disease outcome of a subject with a stage 0 cancer. In some cases, a method may predict the disease outcome of a subject with a stage I cancer. In some cases, a method may predict the disease outcome of a subject with a stage IA cancer. In some cases, a method may predict the disease outcome of a subject with a stage IB cancer. In some cases, a method may predict the disease outcome of a subject with a stage IIA cancer. In some cases, a method may predict the disease outcome of a subject with a stage IIB cancer. In some cases, a method may predict the disease outcome of a subject with a stage IIIA cancer. In some cases, a method may predict the disease outcome of a subject with a stage IIIB cancer. In some cases, a method may predict the disease outcome of a subject with a stage IIIC cancer. In some cases, a method may predict the disease outcome of a subject with a stage IV cancer.

In some cases, a method may predict the disease outcome of a subject with a stage 0 ovarian cancer, a stage I ovarian cancer, a stage II ovarian cancer, a stage III ovarian cancer, a stage IV ovarian cancer. In some cases, a method may predict the disease outcome of a subject with a stage 0 ovarian cancer. In some cases, a method may predict the disease outcome of a subject with a stage I ovarian cancer. In some cases, a method may predict the disease outcome of a subject with a stage IA ovarian cancer. In some cases, a method may predict the disease outcome of a subject with a stage IB ovarian cancer. In some cases, a method may predict the disease outcome of a subject with a stage IIA ovarian cancer. In some cases, a method may predict the disease outcome of a subject with a stage IIB ovarian cancer. In some cases, a method may predict the disease outcome of a subject with a stage IIIA ovarian cancer. In some cases, a method may predict the disease outcome of a subject with a stage IIIB ovarian cancer. In some cases, a method may predict the disease outcome of a subject with a stage IIIC ovarian cancer. In some cases, a method may predict the disease outcome of a subject with a stage IV ovarian cancer.

In some cases, a cancer may also comprise a breast cancer, a colorectal cancer, a lung cancer, an ovarian cancer, a pancreatic cancer, or a combination thereof. In some cases, a cancer may comprise at least two of a breast cancer, a colorectal cancer, a lung cancer, an ovarian cancer, and a pancreatic cancer. In some cases, a cancer may comprises at least two of a liver cancer, a bladder cancer, a pancreatic cancer, a lung cancer, or prostate cancer. In some cases, a cancer may comprise at least three of a breast cancer, a colorectal cancer, a lung cancer, an ovarian cancer, and a pancreatic cancer. In some cases, a cancer may comprise at least four of a breast cancer, a colorectal cancer, a lung cancer, an ovarian cancer, and a pancreatic cancer. In some cases, a cancer may comprise a breast cancer, a colorectal cancer, a lung cancer, an ovarian cancer, and a pancreatic cancer. In some cases, a cancer may comprise a breast cancer. In some cases, a cancer may comprise a colorectal cancer. In some cases, a cancer may comprise a lung cancer. In some cases, a cancer may comprise an ovarian cancer. In some cases, a cancer may comprise a pancreatic cancer. A cancer can also comprise lymphoma, blastoma, sarcoma, leukemia, squamous cell cancer, cancer of the peritoneum, hepatocellular cancer, gastric or stomach cancer, glioblastoma, cervical cancer, liver cancer, bladder cancer, gallbladder cancer, colon cancer, endometrial or uterine carcinoma, salivary gland carcinoma, kidney or renal cancer, renal cell carcinoma, prostate cancer, vulval cancer, thyroid cancer, head and neck cancer.

In some cases, a lung cancer type may comprise small cell lung cancer (SCLC), non-small cell lung cancer (NSCLC), lung carcinoid tumors, adenoid cystic carcinomas, lymphomas, or sarcomas. In some instances, a method may predict the disease outcome of a subject with two of SCLC, NSCLC, lung carcinoid tumors, adenoid cystic carcinomas, lymphomas, and sarcomas. In some instances, a method may predict the disease outcome of a subject with three of SCLC, NSCLC, lung carcinoid tumors, adenoid cystic carcinomas, lymphomas, and sarcomas. In some instances, a method may predict the disease outcome of a subject with four of SCLC, NSCLC, lung carcinoid tumors, adenoid cystic carcinomas, lymphomas, and sarcomas. In some instances, a method may predict the disease outcome of a subject with five of SCLC, NSCLC, lung carcinoid tumors, adenoid cystic carcinomas, lymphomas, and sarcomas. In some instances, a method may predict the disease outcome of a subject with SCLC, NSCLC, lung carcinoid tumors, adenoid cystic carcinomas, lymphomas, and sarcomas. In some instances, a method may predict the disease outcome of a subject with SCLC. In some instances, a method may predict the disease outcome of a subject with NSCLC. In some instances, a method may predict the disease outcome of a subject with lung carcinoid tumors. In some instances, a method may predict the disease outcome of a subject with sarcomas. In some instances, a method may predict the disease outcome of a subject with adenoid cystic carcinomas. In some instances, a method may predict the disease outcome of a subject with lymphomas.

In some instances, a NSCLC subtype may comprise adenocarcinoma of the lung, squamous carcinoma of the lung, large cell (undifferentiated) carcinoma, adenosquamous carcinoma, sarcomatoid carcinoma, or any combinations thereof. In some instances, a method may predict the disease outcome of a subject with two of adenocarcinoma of the lung, squamous carcinoma of the lung, large cell (undifferentiated) carcinoma, adenosquamous carcinoma, and sarcomatoid carcinoma. In some instances, a method may predict the disease outcome of a subject with three of adenocarcinoma of the lung, squamous carcinoma of the lung, large cell (undifferentiated) carcinoma, adenosquamous carcinoma, and sarcomatoid carcinoma. In some instances, a method may predict the disease outcome of a subject with four of adenocarcinoma of the lung, squamous carcinoma of the lung, large cell (undifferentiated) carcinoma, adenosquamous carcinoma, and sarcomatoid carcinoma. In some instances, a method may predict the disease outcome of a subject with adenocarcinoma of the lung, squamous carcinoma of the lung, large cell (undifferentiated) carcinoma, adenosquamous carcinoma, and sarcomatoid carcinoma.

In some instances, a method may predict the disease outcome of a subject with adenocarcinoma of the lung. In some instances, a method may predict the disease outcome of a subject with squamous carcinoma of the lung. In some instances, a method may predict the disease outcome of a subject with large cell (undifferentiated) carcinoma. In some instances, a method may predict the disease outcome of a subject with adenosquamous carcinoma. In some instances, a method may predict the disease outcome of a subject with and sarcomatoid carcinoma.

Colorectal cancer may comprise colorectal adenocarcinoma, gastrointestinal carcinoid tumors, primary colorectal lymphomas, gastrointestinal stromal tumors, leiomyosarcomas, squamous cell carcinomas, familial adenomatous polyposis, or a combination thereof. Colorectal adenocarcinoma may comprise mucinous adenocarcinoma or signet ring cell adenocarcinoma. Pancreatic cancer may comprise exocrine (nonendocrine) pancreatic cancer, neuroendocrine pancreatic cancer, or benign precancerous lesions. Exocrine (nonendocrine) pancreatic cancer can comprise pancreatic adenocarcinoma, pancreatic squamous cell carcinoma, pancreatic adenosquamous carcinoma, pancreatic colloid carcinoma.

In other cases, gastric cancer may include gastrointestinal cancer or gastrointestinal stromal cancer). Melanoma can comprise superficial spreading melanoma, lentigo malignant melanoma, acral lentiginous melanomas, nodular melanomas. Lymphoma may comprise B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, or AIDS-related lymphoma. Leukemia may comprise chronic lymphocytic leukemia (CLL), acute lymphoblastic leukemia (ALL), Hairy cell leukemia, multiple myeloma, acute myeloid leukemia (AML) or chronic myeloblastic leukemia.

In some cases, a method may predict the disease outcome of a subject with a stage 0 lung cancer, a stage I lung cancer, a stage II lung cancer, a stage III lung cancer, a stage IV lung cancer. In some cases, a method may predict the disease outcome of a subject with a stage 0 lung cancer. In some cases, a method may predict the disease outcome of a subject with a stage I lung cancer. In some cases, a method may predict the disease outcome of a subject with a stage IA lung cancer. In some cases, a method may predict the disease outcome of a subject with a stage IB lung cancer. In some cases, a method may predict the disease outcome of a subject with a stage IIA lung cancer. In some cases, a method may predict the disease outcome of a subject with a stage IIB lung cancer. In some cases, a method may predict the disease outcome of a subject with a stage IIIA lung cancer. In some cases, a method may predict the disease outcome of a subject with a stage IIIB lung cancer. In some cases, a method may predict the disease outcome of a subject with a stage IIIC lung cancer. In some cases, a method may predict the disease outcome of a subject with a stage IV lung cancer.

In some cases, a method may predict the disease outcome of a subject with a stage 0 breast cancer, a stage I breast cancer, a stage II breast cancer, a stage III breast cancer, a stage IV breast cancer. In some cases, a method may predict the disease outcome of a subject with a stage 0 breast cancer. In some cases, a method may predict the disease outcome of a subject with a stage I breast cancer. In some cases, a method may predict the disease outcome of a subject with a stage IA breast cancer. In some cases, a method may predict the disease outcome of a subject with a stage IB breast cancer. In some cases, a method may predict the disease outcome of a subject with a stage IIA breast cancer. In some cases, a method may predict the disease outcome of a subject with a stage IIB breast cancer. In some cases, a method may predict the disease outcome of a subject with a stage IIIA breast cancer. In some cases, a method may predict the disease outcome of a subject with a stage IIIB breast cancer. In some cases, a method may predict the disease outcome of a subject with a stage IIIC breast cancer. In some cases, a method may predict the disease outcome of a subject with a stage IV breast cancer.

In some cases, a method may predict the disease outcome of a subject with a stage 0 pancreatic cancer, a stage I pancreatic cancer, a stage II pancreatic cancer, a stage III pancreatic cancer, a stage IV pancreatic cancer. In some cases, a method may predict the disease outcome of a subject with a stage 0 pancreatic cancer. In some cases, a method may predict the disease outcome of a subject with a stage I pancreatic cancer. In some cases, a method may predict the disease outcome of a subject with a stage IA pancreatic cancer. In some cases, a method may predict the disease outcome of a subject with a stage IB pancreatic cancer. In some cases, a method may predict the disease outcome of a subject with a stage IIA pancreatic cancer. In some cases, a method may predict the disease outcome of a subject with a stage IIB pancreatic cancer. In some cases, a method may predict the disease outcome of a subject with a stage IIIA pancreatic cancer. In some cases, a method may predict the disease outcome of a subject with a stage IIIB pancreatic cancer. In some cases, a method may predict the disease outcome of a subject with a stage IIIC pancreatic cancer. In some cases, a method may predict the disease outcome of a subject with a stage IV pancreatic cancer.

In some cases, a method may predict the disease outcome of a subject with a stage 0 colorectal cancer, a stage I colorectal cancer, a stage II colorectal cancer, a stage III colorectal cancer, a stage IV colorectal cancer. In some cases, a method may predict the disease outcome of a subject with a stage 0 colorectal cancer. In some cases, a method may predict the disease outcome of a subject with a stage I colorectal cancer. In some cases, a method may predict the disease outcome of a subject with a stage IA colorectal cancer. In some cases, a method may predict the disease outcome of a subject with a stage IB colorectal cancer. In some cases, a method may predict the disease outcome of a subject with a stage IIA colorectal cancer. In some cases, a method may predict the disease outcome of a subject with a stage IIB colorectal cancer. In some cases, a method may predict the disease outcome of a subject with a stage IIIA colorectal cancer. In some cases, a method may predict the disease outcome of a subject with a stage IIIB colorectal cancer. In some cases, a method may predict the disease outcome of a subject with a stage IIIC colorectal cancer. In some cases, a method may predict the disease outcome of a subject with a stage IV colorectal cancer.

### Micro-Ribonucleic Acids

In some instances, the method described herein may predict the clinical or disease outcome of a subject with cancer using an index. The index may be derived from a nucleic acid of a subject. The nucleic acid may comprise deoxyribose nucleic acids (DNAs) or ribonucleic acids (RNAs). In some instances, a nucleic acid may be a nucleic acid molecule. In some cases, a nucleic acid may be a species/type of nucleic acids. A nucleic acid molecule may comprise one or more modified nucleotides, such as methylated nucleotides and nucleotide analogs. In some cases, a nucleic acid molecule may comprise a polymeric form of nucleotides. In some cases, a nucleic acid molecule may comprise a polynucleotide. In some cases, a nucleic acid molecule may comprise a modified polynucleotide. In some cases, a nucleic acid molecule may comprise a canonical or non-canonical nucleotide. A canonical nucleotide may comprise adenosine (A), cytosine (C), guanine (G), thymine (T), uracil (U), or variants thereof. In some cases, a nucleic acid may be single-stranded, double-stranded or triple stranded. In some cases, a nucleic acid may be single-stranded. In some cases, a nucleic acid may be double-stranded. In some cases, a nucleic acid may be single-stranded and double-stranded. The index may be derived from RNAs. The index may be derived from DNAs.

The RNAs for deriving an index may comprise an extracellular RNAs. The RNA for deriving an index may comprise an RNA not encompassed by a cell immediately prior to when the RNA is being extracted. Such an RNA may also be referred to as a cell-free RNA. For example, the cell-free RNA may not be encompassed by a plasma membrane of a cell. In other cases, the cell-free RNA may not be encompassed or residing within the cytoplasm of a cell. The cell-free RNA may reside outside of a cell. The cell may comprise a living or a viable cell. The cell may comprise an intact cell.

A cell-free RNA or an extracellular RNA may be secreted by a cell. In some cases, the RNA may be encompassed by a lipid membrane. In some cases, the RNA may be encompassed by a vesicle. In some cases, the RNA may be encompassed by an extracellular vesicle or extracellular membrane vesicle. In some cases, the RNA may be encompassed by an exosome. The RNA may be circulating within the bodily fluid of a subject. The RNA may be circulating within the blood or serum of a subject. The RNA may be secreted by a viable or intact cell. The RNA may also be released by a dying, non-viable, non-healthy, or non-intact cell.

The RNA may comprise a non-coding RNA. In some cases, the RNA may comprise a small non-coding RNA. In some cases, the length of the non-coding RNA may be at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 100 nucleotides (nt) in length. the length of the non-coding RNA may be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 100 nt in length. Non-coding RNAs for generating an index may comprise a micro-ribonucleic acid (miRNA or microRNA), transfer ribonucleic acid (tRNA), a long non-coding RNA (lncRNA), a ribosomal ribonucleic acid (rRNA), a small nuclear RNA (snRNA), a piwi-interacting RNA (piRNA), a small nucleolar RNA (snoRNA), an extracellular RNA (exRNA), a small cajal body-specific RNA (scaRNA), a silencing ribonucleic acid (siRNA), a YRNA (small noncoding RNA), a heterogeneous nuclear RNA (HnRNA), or endless/circular RNA (eRNA).

In some instances, the RNA for generating an index may comprise a miRNA. In some instances, miRNAs can be endogenous. In some instances, miRNAs can be single-stranded. In some instances, miRNAs can be double-stranded. In some cases, the miRNAs can comprise both single-stranded and double stranded region. In some cases, the miRNAs can comprise a step-loop region. In some instances, miRNAs can be non-coding small RNAs. In some instances, miRNAs can regulate target gene expression. In some cases, the miRNA may comprise a sequence complementary to a sequence of a messenger RNA (mRNA). When base-pairing with the mRNA, the miRNA may inhibit, decrease, or prevent the translation of the mRNA. In other cases, an miRNA may comprise a sequence complementary to a sequence of a promoter or enhancer region of a gene. When base-pairing with the promoter or enhancer region of a gene (e.g., the miRNA base-paired with the DNA of the gene promoter or enhancer), the miRNA may activate the expression or transcription of the gene.

In some cases, the miRNA may be at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 100 nucleotides (nt) in length. The miRNA may be at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 100 nt in length. In some cases, the miRNA may have about 22 nt in length.

In some instances, a miRNA may be a cell-free miRNA. In some cases, the cell-free miRNA may not be encompassed by a plasma membrane of a cell. In other cases, the cell-free miRNA may not be encompassed or residing within the cytoplasm of a cell. The cell-free miRNA may reside outside of a cell. A cell-free miRNA or an extracellular miRNA may be secreted by a cell. In some cases, the miRNA may be encompassed by a lipid membrane. In some cases, the miRNA may be encompassed by a vesicle. In some cases, the miRNA may be encompassed by an extracellular vesicle or extracellular membrane vesicle. In some cases, the miRNA may be encompassed by an exosome. The miRNA may be circulating within the bodily fluid of a subject. The miRNA may be circulating within the blood or serum of a subject. In instances, the miRNAs can be secreted from cells in extracellular vesicles and can mediate cell-to-cell communication in the local and distant microenvironment.

In some cases, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 500, 1000, 2000, 5000, 10000 or more miRNAs can be used to generate an index. In some cases, at least about 1 miRNA can be used to generate an index. In some cases, at least about 2 miRNAs can be used to generate an index. In some cases, at least about 3 miRNAs can be used to generate an index. In some cases, at least about 4 miRNAs can be used to generate an index. In some cases, at least about 5 miRNAs can be used to generate an index. In some cases, at least about 6 miRNAs can be used to generate an index. In some cases, at least about 7 miRNAs can be used to generate an index. In some cases, at least about 8 miRNAs can be used to generate an index. In some cases, at least about 9 miRNAs can be used to generate an index. In some cases, at least about 10 miRNAs can be used to generate an index. In some cases, at least about 11 miRNAs can be used to generate an index. In some cases, at least about 12 miRNAs can be used to generate an index. In some cases, at least about 13 miRNAs can be used to generate an index. In some cases, at least about 14 miRNAs can be used to generate an index. In some cases, at least about 15 miRNAs can be used to generate an index. In some cases, at least about 16 miRNAs can be used to generate an index. In some cases, at least about 17 miRNAs can be used to generate an index. In some cases, at least about 18 miRNAs can be used to generate an index. In some cases, at least about 19 miRNAs can be used to generate an index. In some cases, at least about 20 miRNAs can be used to generate an index. In some cases, at least about 21 miRNAs can be used to generate an index. In some cases, at least about 22 miRNAs can be used to generate an index. In some cases, at least about 23 miRNAs can be used to generate an index. In some cases, at least about 24 miRNAs can be used to generate an index. In some cases, at least about 25 miRNAs can be used to generate an index. In some cases, at least about 26 miRNAs can be used to generate an index. In some cases, at least about 27 miRNAs can be used to generate an index. In some cases, at least about 28 miRNAs can be used to generate an index. In some cases, at least about 29 miRNAs can be used to generate an index. In some cases, at least about 30 miRNAs can be used to generate an index. In some cases, at least about 31 miRNAs can be used to generate an index. In some cases, at least about 32 miRNAs can be used to generate an index. In some cases, at least about 33 miRNAs can be used to generate an index. In some cases, at least about 34 miRNAs can be used to generate an index. In some cases, at least about 35 miRNAs can be used to generate an index. In some cases, at least about 36 miRNAs can be used to generate an index. In some cases, at least about 37 miRNAs can be used to generate an index. In some cases, at least about 38 miRNAs can be used to generate an index. In some cases, at least about 39 miRNAs can be used to generate an index. In some cases, at least about 40 miRNAs can be used to generate an index. In some cases, at least about 41 miRNAs can be used to generate an index. In some cases, at least about 42 miRNAs can be used to generate an index. In some cases, at least about 43 miRNAs can be used to generate an index. In some cases, at least about 44 miRNAs can be used to generate an index. In some cases, at least about 45 miRNAs can be used to generate an index. In some cases, at least about 46 miRNAs can be used to generate an index. In some cases, at least about 47 miRNAs can be used to generate an index. In some cases, at least about 48 miRNAs can be used to generate an index. In some cases, at least about 49 miRNAs can be used to generate an index. In some cases, at least about 50 miRNAs can be used to generate an index. In some cases, at least about 100 miRNAs can be used to generate an index. In some cases, at least about 150 miRNAs can be used to generate an index. In some cases, at least about 200 miRNAs can be used to generate an index. In some cases, at least about 500 miRNAs can be used to generate an index. In some cases, at least about 1000 miRNAs can be used to generate an index. In some cases, at least about 2000 miRNAs can be used to generate an index. In some cases, at least about 5000 miRNAs can be used to generate an index. In some cases, at least about 10000 miRNAs can be used to generate an index. In some cases, at least about more than 10000 miRNAs can be used to generate an index.

In some cases, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 500, 1000, 2000, 5000, or 10000 miRNAs can be used to generate an index. In some cases, at most about 1 miRNA can be used to generate an index. In some cases, at most about 2 miRNAs can be used to generate an index. In some cases, at most about 3 miRNAs can be used to generate an index. In some cases, at most about 4 miRNAs can be used to generate an index. In some cases, at most about 5 miRNAs can be used to generate an index. In some cases, at most about 6 miRNAs can be used to generate an index. In some cases, at most about 7 miRNAs can be used to generate an index. In some cases, at most about 8 miRNAs can be used to generate an index. In some cases, at most about 9 miRNAs can be used to generate an index. In some cases, at most about 10 miRNAs can be used to generate an index. In some cases, at most about 11 miRNAs can be used to generate an index. In some cases, at most about 12 miRNAs can be used to generate an index. In some cases, at most about 13 miRNAs can be used to generate an index. In some cases, at most about 14 miRNAs can be used to generate an index. In some cases, at most about 15 miRNAs can be used to generate an index. In some cases, at most about 16 miRNAs can be used to generate an index. In some cases, at most about 17 miRNAs can be used to generate an index. In some cases, at most about 18 miRNAs can be used to generate an index. In some cases, at most about 19 miRNAs can be used to generate an index. In some cases, at most about 20 miRNAs can be used to generate an index. In some cases, at most about 21 miRNAs can be used to generate an index. In some cases, at most about 22 miRNAs can be used to generate an index. In some cases, at most about 23 miRNAs can be used to generate an index. In some cases, at most about 24 miRNAs can be used to generate an index. In some cases, at most about 25 miRNAs can be used to generate an index. In some cases, at most about 26 miRNAs can be used to generate an index. In some cases, at most about 27 miRNAs can be used to generate an index. In some cases, at most about 28 miRNAs can be used to generate an index. In some cases, at most about 29 miRNAs can be used to generate an index. In some cases, at most about 30 miRNAs can be used to generate an index. In some cases, at most about 31 miRNAs can be used to generate an index. In some cases, at most about 32 miRNAs can be used to generate an index. In some cases, at most about 33 miRNAs can be used to generate an index. In some cases, at most about 34 miRNAs can be used to generate an index. In some cases, at most about 35 miRNAs can be used to generate an index. In some cases, at most about 36 miRNAs can be used to generate an index. In some cases, at most about 37 miRNAs can be used to generate an index. In some cases, at most about 38 miRNAs can be used to generate an index. In some cases, at most about 39 miRNAs can be used to generate an index. In some cases, at most about 40 miRNAs can be used to generate an index. In some cases, at most about 41 miRNAs can be used to generate an index. In some cases, at most about 42 miRNAs can be used to generate an index. In some cases, at most about 43 miRNAs can be used to generate an index. In some cases, at most about 44 miRNAs can be used to generate an index. In some cases, at most about 45 miRNAs can be used to generate an index. In some cases, at most about 46 miRNAs can be used to generate an index. In some cases, at most about 47 miRNAs can be used to generate an index. In some cases, at most about 48 miRNAs can be used to generate an index. In some cases, at most about 49 miRNAs can be used to generate an index. In some cases, at most about 50 miRNAs can be used to generate an index. In some cases, at most about 100 miRNAs can be used to generate an index. In some cases, at most about 150 miRNAs can be used to generate an index. In some cases, at most about 200 miRNAs can be used to generate an index. In some cases, at most about 500 miRNAs can be used to generate an index. In some cases, at most about 1000 miRNAs can be used to generate an index. In some cases, at most about 2000 miRNAs can be used to generate an index. In some cases, at most about 5000 miRNAs can be used to generate an index. In some cases, at most about 10000 miRNAs can be used to generate an index.

In some cases, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 500, 1000, 2000, 5000, or 10000 miRNAs can be used to generate an index. In some cases, 1 miRNA can be used to generate an index. In some cases, 2 miRNAs can be used to generate an index. In some cases, 3 miRNAs can be used to generate an index. In some cases, 4 miRNAs can be used to generate an index. In some cases, 5 miRNAs can be used to generate an index. In some cases, 6 miRNAs can be used to generate an index. In some cases, 7 miRNAs can be used to generate an index. In some cases, 8 miRNAs can be used to generate an index. In some cases, 9 miRNAs can be used to generate an index. In some cases, 10 miRNAs can be used to generate an index. In some cases, 11 miRNAs can be used to generate an index. In some cases, 12 miRNAs can be used to generate an index. In some cases, 13 miRNAs can be used to generate an index. In some cases, 14 miRNAs can be used to generate an index. In some cases, 15 miRNAs can be used to generate an index. In some cases, 16 miRNAs can be used to generate an index. In some cases, 17 miRNAs can be used to generate an index. In some cases, 18 miRNAs can be used to generate an index. In some cases, 19 miRNAs can be used to generate an index. In some cases, 20 miRNAs can be used to generate an index. In some cases, 21 miRNAs can be used to generate an index. In some cases, 22 miRNAs can be used to generate an index. In some cases, 23 miRNAs can be used to generate an index. In some cases, 24 miRNAs can be used to generate an index. In some cases, 25 miRNAs can be used to generate an index. In some cases, 26 miRNAs can be used to generate an index. In some cases, 27 miRNAs can be used to generate an index. In some cases, 28 miRNAs can be used to generate an index. In some cases, 29 miRNAs can be used to generate an index. In some cases, 30 miRNAs can be used to generate an index. In some cases, 31 miRNAs can be used to generate an index. In some cases, 32 miRNAs can be used to generate an index. In some cases, 33 miRNAs can be used to generate an index. In some cases, 34 miRNAs can be used to generate an index. In some cases, 35 miRNAs can be used to generate an index. In some cases, 36 miRNAs can be used to generate an index. In some cases, 37 miRNAs can be used to generate an index. In some cases, 38 miRNAs can be used to generate an index. In some cases, 39 miRNAs can be used to generate an index. In some cases, 40 miRNAs can be used to generate an index. In some cases, 41 miRNAs can be used to generate an index. In some cases, 42 miRNAs can be used to generate an index. In some cases, 43 miRNAs can be used to generate an index. In some cases, 44 miRNAs can be used to generate an index. In some cases, 45 miRNAs can be used to generate an index. In some cases, 46 miRNAs can be used to generate an index. In some cases, 47 miRNAs can be used to generate an index. In some cases, 48 miRNAs can be used to generate an index. In some cases, 49 miRNAs can be used to generate an index. In some cases, 50 miRNAs can be used to generate an index. In some cases, 100 miRNAs can be used to generate an index. In some cases, 150 miRNAs can be used to generate an index. In some cases, 200 miRNAs can be used to generate an index. In some cases, 500 miRNAs can be used to generate an index. In some cases, 1000 miRNAs can be used to generate an index. In some cases, 2000 miRNAs can be used to generate an index. In some cases, 5000 miRNAs can be used to generate an index. In some cases, 10000 miRNAs can be used to generate an index.

In some instances, the miRNA used to generate an index can comprise miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, miR-6805-5p, or a combination thereof. In some instances, the miRNA used to generate an index can comprise at least about 1 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 2 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 3 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 4 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 5 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 6 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 7 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 8 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 9 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 10 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 11 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 12 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 13 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 14 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 15 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 16 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 17 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 18 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 19 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at least about 20 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p.

In some instances, the miRNA used to generate an index can comprise at most about 1 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 2 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 3 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 4 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 5 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 6 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 7 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 8 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 9 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 10 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 11 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 12 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 13 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 14 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 15 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 16 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 17 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 18 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 19 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise at most about 20 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p.

In some instances, the miRNA used to generate an index can comprise 1 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 2 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 3 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 4 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 5 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 6 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 7 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 8 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 9 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 10 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 11 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 12 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 13 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 14 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 15 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 16 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 17 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 18 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 19 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p. In some instances, the miRNA used to generate an index can comprise 20 of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, and miR-6805-5p.

In some cases, at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, or 210 of miRNAs described in TABLE 1 can be used to generate an index. In some cases, at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, or 210 of miRNAs described in TABLE 1 can be used to generate an index. In some cases, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, or 210 of miRNAs described in TABLE 1 can be used to generate an index.

In some instances, the miRNA used to generate an index can comprise miR-187-5p. In some instances, the miRNA used to generate an index can comprise miR-6870-5p. In some instances, the miRNA used to generate an index can comprise miR-1908-5p. In some instances, the miRNA used to generate an index can comprise miR-6727-5p. In some instances, the miRNA used to generate an index can comprise miR-711. In some instances, the miRNA used to generate an index can comprise miR-1229-5p. In some instances, the miRNA used to generate an index can comprise miR-1914-3p. In some instances, the miRNA used to generate an index can comprise miR-4513. In some instances, the miRNA used to generate an index can comprise miR-4656. In some instances, the miRNA used to generate an index can comprise miR-4787-3p. In some instances, the miRNA used to generate an index can comprise miR-6787-5p. In some instances, the miRNA used to generate an index can comprise miR-6850-5p. In some instances, the miRNA used to generate an index can comprise miR-7107-5p. In some instances, the miRNA used to generate an index can comprise miR-7150. In some instances, the miRNA used to generate an index can comprise miR-150-3p. In some instances, the miRNA used to generate an index can comprise miR-3195. In some instances, the miRNA used to generate an index can comprise miR-7704. In some instances, the miRNA used to generate an index can comprise miR-365a-5p. In some instances, the miRNA used to generate an index can comprise miR-4730. In some instances, the miRNA used to generate an index can comprise miR-671-5p. In some instances, the miRNA used to generate an index can comprise miR-6805-5p.

In some instances, the miRNA used to generate an index can comprise at least two miRNAs. In some instances, the at least two miRNAs can comprise miR-187-5p, miR-6870-5p, miR-1908-5p, miR-6727-5p, or any combination thereof. In some instances, the at least two miRNAs can comprise miR-187-5p. In some instances, the at least two miRNAs can comprise miR-6870-5p. In some instances, the at least two miRNAs can comprise miR-1908-5p. In some instances, the at least two miRNAs can comprise miR-6727-5p. In some instances, the at least two miRNAs can comprise 187-5p and miR-6870-5p. In some instances, the at least two miRNAs can comprise miR-187-5p, miR-6870-5p, and miR-1908-5p. In some instances, the at least two miRNAs can comprise miR-187-5p, miR-6870-5p, and miR-6727-5p. In some instances, the at least two miRNAs can comprise miR-187-5p, miR-6870-5p, miR-1908-5p, and miR-6727-5p. In some instances, the at least two miRNAs can comprise miR-150-3p, miR-3195, miR-7704, or any combination thereof. In some instances, the at least two miRNAs can comprise miR-150-3p. In some instances, the at least two miRNAs can comprise miR-3195. In some instances, the at least two miRNAs can comprise miR-7704. In some instances, the at least two miRNAs can comprise miR-150-3p, miR-3195 and miR-7704. In some instances, the miRNAs used to generate an index can comprise a miRNA thereof described in EXAMPLE 1 or listed in TABLE 1. In some cases, a combination of a miRNA can be used to generate as index, wherein the combination can be identified based on the methods described in EXAMPLE 2 and/or EXAMPLE 3. Other methods for identifying the combination of miRNA for generating an index are described elsewhere in this disclosure.

In some instances, a miRNA for generating an index may be identified from a dataset of a database. In some cases, the database can comprise a database of National Center for Biotechnology Information (NCBI). In some cases, a database may comprise Gene Expression Omnibus (GEO). In some cases, a dataset of a database may have a identifying number. For example, an identifying number of a dataset of GEO may be an accession number. In some cases, a miRNA can be any one of the miRNAs described in the dataset with an accession number GSE106817 of GEO of NCBI. A miRNA described in other datasets or databases can also be used to generate an index using methods described here.

In some cases, a composition may comprise a probe to any miRNAs described herein. The probe may have a sequence of the miRNA. The probe may also have a sequence complementary to the miRNA. Such a probe may comprise a primer, a hybridization probe, a probe for microarray, a probe for sequencing of the miRNA, or a combination thereof. A kit may comprise the compositions. A kit may also comprise compositions for practicing the isolation and identification of miRNAs described herein.

Levels of miRNAs

In some instances, the method described herein may comprise generating an index. In some instances, an index can comprise a value that is predictive of the clinical or disease outcome of a subject with cancer. In some instances, an index can be generated using levels of miRNA. A level of miRNA may comprise the expression level of the miRNA.

The expression level of the miRNA can comprise the amount of the miRNA measured in a sample of a subject. In some instances, the expression level of the miRNA can be used to generate the index. In some instances, the level of miRNA can be measured as a concentration level. The concentration level can be expressed as weight/volume. In some instances, the level of miRNA can be measured in milligram/milliliter (mg/ml). In some instances, the level of miRNA can be measured in microgram/milliliter (μg/ml). In some instances, the level of miRNA can be measured in nanogram/milliliter (ng/ml). In some instances, the level of miRNA can be measured in picogram/milliliter (pg/ml). In some instances, the level of miRNA can be measured in microgram/microliter (μg/μl). In some instances, the level of miRNA can be measured in nanogram/microliter (ng/μl). In some instances, the level of miRNA can be measured in picogram/microliter (pg/μl). The concentration may be the level of miRNA in a subject. The concentration may be the level of miRNA in a cell-free sample of a subject. The concentration may be the level of miRNA in a bodily fluid of a subject. The concentration can also be expressed as the molar form. In some instances, the level of miRNA can also be measured as weight. For example, the weight level of a miRNA can comprise mg, μg, ng, or pg. In some instances, the level of miRNA can also be measured as numbers of molecules. One example unit for numbers of molecules is mole.

In some instances, the index can be generated using levels of miRNA, expression levels of miRNA, normalized levels (as described herein) of miRNA, or a combination thereof. In some instances, the index can be generated using levels of miRNA. In some instances, the index can be generated using expression levels of miRNA. In some instances, the index can be generated using normalized levels of miRNA.

In some instances, the miRNA levels can be normalized by dividing the specific miRNA level measured by the level of a control (e.g., a housekeeping gene). For example, a control may be a gene expressed at a constant level. In some instances, the control gene can be a gene that is constantly expressed in cells. A constant expression may comprise a level (e.g., expression level) of a gene or gene transcript that is constant among cell types or conditions (e.g., disease conditions, health states of a cell or a subject, or treatment conditions) In some instances, the control gene can be a gene transcribed as a coding RNA (e.g., actin, Glyceraldehyde-3-Phsopate Dehydrogenase (GAPDH), or ubiquitin). The mRNA level of the coding gene may the level of the control. In some instances, the control can be comprise a miRNA. The control miRNA can comprise a housekeeping miRNA. A housekeeping miRNA may be a miRNA with a constant expression level. The housekeeping miRNA may comprise miR-151a-5p, miR-27b-3p, or miR-103a-3p. The level of a control may comprise any levels described herein. Normalized levels may refer to relative levels.

In some instances, a level may comprise a normalized level. In some cases, the level of miRNA can be normalized using a statistical normalization method. In some instances, the statistical normalization method can be a Z-score transformation. In some instances, the statistical normalization method can be a range transformation. In some instances, the statistical normalization method can be a proportion transformation. In some instances, the statistical normalization method can be the interquartile range. In some instances, the statistical normalization can be a linear scaling. In some instances, the statistical normalization method can be clipping. In some instances, the statistical normalization method can be log scaling. In some instances, the statistical normalization method can be a linear normalization.

In some cases, the normalized level may comprise a parametric value of a level of miRNA measured in multiple instances (e.g., in multiple replicates). The replicate may comprise a biological replicate. Replicates may comprise measurements in different biological samples. The normalized level or parametric value of a level of miRNA may comprise a parameter of a measurement of levels of miRNAs. A normalized level of miRNA may comprise a mean, mode, median, maximum, minimum, range, first quartile, second quartile, third quartile, or fourth quartile value of the levels of miRNA. In some cases, a normalized level of miRNA may comprise a mean of levels of the miRNA. In some cases, a normalized level of miRNA may comprise a mode of levels or expression levels of the miRNA. In some cases, a normalized level of miRNA may comprise a median of levels or expression levels of the miRNA. In some cases, a normalized level of miRNA may comprise a maximum of levels or expression levels of the miRNA. In some cases, a normalized level of miRNA may comprise a minimum of levels or expression levels of the miRNA. In some cases, a normalized level of miRNA may comprise a range of levels or expression levels of the miRNA. In some cases, a normalized level of miRNA may comprise a first quartile of levels or expression levels of the miRNA. In some cases, a normalized level of miRNA may comprise a second quartile of levels or expression levels of the miRNA. In some cases, a normalized level of miRNA may comprise a third quartile of levels or expression levels of the miRNA. In some cases, a normalized level of miRNA may comprise a fourth quartile value of levels or expression levels of the miRNA. A normalized level may be a level derived from an addition, subtraction, multiplication, division, or a combination thereof of more than one measurement or parameter. In other cases, the normalized level may comprise any parameters described herein of an expression distribution of an entity (e.g., a gene, gene transcript, RNA, or a miRNA). In some cases, a normalized level of miRNA may be expressed as a rank among a list of miRNAs. The rank may be based on the level, expression level, or normalized of miRNAs.

In some cases, a level, expression level, or normalized level of miRNA may be converted to a threshold level of miRNA. In some cases, a threshold level of miRNA may also be used to predict an outcome of a disease. In some instances, the threshold level miRNA may comprise a level of the miRNA in a in a subject positive for a cancer. In some instances, the threshold level miRNA may comprise a level of the miRNA in a in a subject with a cancer or with a risk of having or developing the cancer. In some instances, the threshold level miRNA may comprise a level of the miRNA in a in a subject negative for a cancer. In some instances, the threshold level miRNA may comprise a level of the miRNA in a in a subject without a cancer or without a risk of having or developing the cancer. The subject may be healthy. In some cases, the threshold level miRNA may comprise a level of the miRNA in a cell, tissue, or subject with a cancer; or a sample derived from a cell, tissue, or subject with a cancer. In some cases, the threshold level miRNA may be a mean, mode, median, maximum, minimum, range, first quartile, second quartile, third quartile, or forth quartile level of the miRNA in a sample derived from a subject with a cancer or with a risk of having or developing the cancer. In some cases, the threshold level miRNA may comprise a level of the miRNA in a cell, tissue, or subject without a cancer; or a sample derived from a cell, tissue, or subject without a cancer. In some cases, the threshold level miRNA may be a mean, mode, median, maximum, minimum, range, first quartile, second quartile, third quartile, or forth quartile level of the miRNA in a sample derived from a subject without a cancer or without a risk of having or developing the cancer.

A threshold level of miRNA may comprise a mean, mode, median, maximum, minimum, range, first quartile, second quartile, third quartile, or fourth quartile value of the measurement. In some cases, a threshold level of miRNA may comprise a mean of levels or normalized levels of the miRNA. In some cases, a threshold level of miRNA may comprise a mode of levels or normalized levels of the miRNA. In some cases, a threshold level of miRNA may comprise a median of levels or normalized levels of the miRNA. In some cases, a threshold level of miRNA may comprise a maximum of levels or normalized levels of the miRNA. In some cases, a threshold level of miRNA may comprise a minimum of levels or normalized levels of the miRNA. In some cases, a threshold level of miRNA may comprise a range of levels or normalized levels of the miRNA. In some cases, a threshold level of miRNA may comprise a first quartile of levels or normalized levels of the miRNA. In some cases, a threshold level of miRNA may comprise a second quartile of levels or normalized levels of the miRNA. In some cases, a threshold level of miRNA may comprise a third quartile of levels or normalized levels of the miRNA. In some cases, a threshold level of miRNA may comprise a fourth quartile value of levels or normalized levels of the miRNA. A threshold level may be a level derived from an addition, subtraction, multiplication, division, or a combination thereof of more than one measurement or parameter. In other cases, the threshold level may comprise any parameters described herein of an expression distribution of an RNA.

A nucleic acid for generating an index or predicting an outcome of a disease may be a biomarker. In some cases, a plurality of threshold levels of miRNA may also be used to predict an outcome of a disease. In some instances, a plurality of threshold levels may refer to a cancer pattern or a cancer biomarker pattern. A biomarker may comprise a measurement of a miRNA level.

### Disease Outcome

In some instances, an index may be used to predict a disease outcome. A disease outcome may comprise an amount of time a subject is alive or not deceased subsequent to being determined to have a disease. A disease outcome may comprise an amount of time a subject is alive or not deceased subsequent to being subjected to a treatment of a disease. A disease outcome may comprise an amount of time a subject is alive or not deceased subsequent to being determined to have a risk of a disease. A disease outcome may comprise an amount of time a subject is alive or not deceased subsequent to being determined to have a cancer. A disease outcome may comprise an amount of time a subject is alive or not deceased subsequent to being subjected to a treatment of a cancer. A disease outcome may comprise an amount of time a subject is alive or not deceased subsequent to being determined to have a risk of a cancer. In some cases, the amount of time a subject is alive or not deceased subsequent to the time of a subject being determined to have a disease or being subjected to a treatment for the disease may comprise overall survival (OS). OS may comprise the amount of time a subject is alive or not deceased subsequent to the time of the subject being determined to have a disease. OS may also comprise the amount of time a subject is alive or not deceased subsequent to the time of the subject being subjected to a treatment for the disease. The disease may comprise any cancers described herein.

A disease outcome may comprise an amount of time a subject lives without a disease worsens. In some cases, the amount of time during or subsequent to a treatment of a disease that a subject is alive or not deceased without the disease worsening may comprise progression-free disease (PFS). A disease may worsen when the subject is suffering from an increasing amount of symptoms or more symptoms. A disease may worsen when the subject is suffering from an increasing intensity of the symptoms or disease. The intensity of a symptom or disease may comprise a measurement of a biomarker of a disease (e.g., a level of the biomarker), an amount of damage of a cell or tissue of a subject resulted from the disease, a feeling of a subject regarding a health stage of the subject, or a combination thereof. A disease may worsen when the subject is subjected to an increasing amount or doses of treatments to maintain an intensity of a symptom or disease. A cancer may worsen when the subject is suffering from an increasing amount of symptoms or more symptoms. A cancer may worsen when the subject is suffering from an increasing intensity of the symptoms or cancer. The intensity of a symptom or cancer may comprise a measurement of a biomarker of a cancer (e.g., a level of the biomarker), an amount of damage of a cell or tissue of a subject resulted from the cancer, a feeling of a subject regarding a health stage of the subject, or a combination thereof. A cancer may worsen when the subject is subjected to an increasing amount or doses of treatments to maintain an intensity of a symptom or cancer. In some cases, the worsening of cancer may comprise a cancer at a stage progressing to a subsequent cancer stage. In some cases, the cancer is worsening if a stage 0 cancer is progressed to a stage I, IA, IB, II, IIA, IIB, IIII, IIIA, IIIB, IIIC, or IV cancer. In some cases, the cancer is worsening if a stage I cancer is progressed to a stage II, IIA, IIB, IIII, IIIA, IIIB, IIIC, or IV cancer. In some cases, the cancer is worsening if a stage IA cancer is progressed to a stage IB, II, IIA, IIB, IIII, IIIA, IIIB, IIIC, or IV cancer. In some cases, the cancer is worsening if a stage IB cancer is progressed to a stage II, IIA, IIB, IIII, IIIA, IIIB, IIIC, or IV cancer. In some cases, the cancer is worsening if a stage II cancer is progressed to a stage IIII, IIIA, IIIB, IIIC, or IV cancer. In some cases, the cancer is worsening if a stage IIA cancer is progressed to a stage IIB, IIII, IIIA, IIIB, IIIC, or IV cancer. In some cases, the cancer is worsening if a stage IIB cancer is progressed to a stage IIII, IIIA, IIIB, IIIC, or IV cancer. In some cases, the cancer is worsening if a stage III cancer is progressed to a stage IV cancer. In some cases, the cancer is worsening if a stage IIIA cancer is progressed to a stage IIIB, IIIC, or IV cancer. In some cases, the cancer is worsening if a stage IIIB cancer is progressed to a stage IIIC or IV cancer. In some cases, the cancer is worsening if a stage IIIC cancer is progressed to a stage IV cancer.

In some cases, OS and PFS can be measured using a survival curve. For example, a survival curve can comprise a Kaplan-Meyer curve.

### Index and Disease Outcome Prediction

In some instances, an index can be generated by computer processing levels of miRNAs in subjects. The computer processing may comprise subjecting a level of a miRNA to a statistical model. The computer processing may comprise subjecting levels of a plurality of miRNAs to a statistical model. A statistical model may comprise a mathematical model that embodies a set of statistical assumptions concerning the generation of sample data. In some instances, the statistical model used to generate the index can be a log-rank test. In some instances, the statistical model used to generate the index can be a linear regression model. A linear regression model can comprise a model that assumes a linear relationship between the input values and the single output variable. In some instances, the statistical model used to generate the index can be an univariate Cox regression model or univariate Cox model. A univariate Cox regression model can comprise a regression model used to determine the association between one input value and an outcome. In some instances, the statistical model used to generate the index can be a multivariate Cox regression model or multivariate Cox model. A multivariate Cox regression model can comprise to a regression model used to determine the association between one or more input values and one or more outcomes. In some instances, the input value can be a level of a miRNA described herein. In some instances, the outcome can be Overall Survival (OS). In some instances, the outcome can be Progression-Free Survival (PFS). In some instances, an index can be generated for overall survival (OS). In some instances, an index can be generated for Progression-Free Survival (PFS).

In some instances, an index can predict a disease outcome. In some instances, an index can detect the presence or absence of a disease. In other instances, an index can predict the disease outcome but not detect the presence or absence of the disease.

In some instances, an index can predict a disease outcome of a cancer. In some instances, an index can predict a disease outcome of an ovarian cancer. In some instances, an index can predict a disease outcome of HGSOC. In some instances, an index can predict a disease outcome of clear cell ovarian carcinoma.

In some instances, an index may comprise a formula. In some cases, a formula may comprise:

Index=Σ[Xₙ*(Yₙ)]

wherein X denotes a derivative value to multiply a level of a miRNA, wherein Y denotes a level of the miRNA, and wherein n denotes one miRNA.

In some cases, Yₙ may comprise a level of any miRNA described herein. For a level of particular miRNA (i.e., Yₙ), Xₙ may comprise a number to multiply Yₙ. In some cases, Xₙ may comprise a number. The number for Xₙ may comprise at least about 0.0000001, 0.000001, 0.00001, 0.0001, 0.001, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 3, 4, 5, 6, 7, 8, 9, 10, 11, 12, 13, 14, 15, 16, 17, 18, 19, 20, 50, 100, 1000 or more. The number for Xₙ may comprise at most about 0.0000001, 0.000001, 0.00001, 0.0001, 0.001, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 3, 4, 5, 6, 7, 8, 9, 10, 11, 12, 13, 14, 15, 16, 17, 18, 19, 20, 50, 100, 1000. The number for Xₙ may comprise about 0.0000001, 0.000001, 0.00001, 0.0001, 0.001, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 3, 4, 5, 6, 7, 8, 9, 10, 11, 12, 13, 14, 15, 16, 17, 18, 19, 20, 50, 100, 1000. The number for Xₙ may comprise 0.0000001, 0.000001, 0.00001, 0.0001, 0.001, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 3, 4, 5, 6, 7, 8, 9, 10, 11, 12, 13, 14, 15, 16, 17, 18, 19, 20, 50, 100, 1000.

A formula of an index may comprise any one of:

$$\begin{matrix}1. Index = 02 \; 18 \times ( miR - 187 - 5 p ) + 0280 \times ( miR - 6870 - 5 p ) ; \\ 2 . Index = 0.148 \times ( miR - 187 - 5 p ) + 0.273 \times ( miR - 6870 - 5 p ) + 0.186 \times ( miR - 1908 - 5 p ) ; \\ 3 . Index = 0.034 \times ( miR - 187 - 5 p ) + 0.236 \times ( miR - 6870 - 5 p ) + 0.5 \; 04 \times ( miR - 6727 - 5 p ) + 0.048 \times ( miR - 1908 - 5 p ) - 0251 \times ( miR - 6870 - 5 p ) ; \\ 4 . Index = 0.031 \times ( miR - 187 - 5 p ) + 0.231 \times ( miR - 6870 - 5 p ) + 0.351 \times ( m i R - 6 \; 727 - 5 p ) ; \\ 5 . Index = 0.463 \times ( miR - 150 - 3 p ) + 1.323 \times ( miR - 3195 ) + 0.636 \times ( m i R - 77 \; 04 ) ; or \\ 6 . Index = 0.399 \times ( miR - 150 - 3 p ) + 1.426 \times ( miR - 3195 ) + 0.48 \times ( m i R - 77 \; 04 ) .\end{matrix}$$

A formula of an index may comprise formula 1. A formula of an index may comprise formula 2. A formula of an index may comprise formula 3. A formula of an index may comprise formula 4. A formula of an index may comprise formula 5. A formula of an index may comprise formula 6. A formula of an index may comprise any two of formula 1, formula 2, formula 3, formula 4, formula 5, and formula 6. A formula of an index may comprise any three of formula 1, formula 2, formula 3, formula 4, formula 5, and formula 6. A formula of an index may comprise any three of formula 1, formula 2, formula 3, formula 4, formula 5, and formula 6. A formula of an index may comprise any four of formula 1, formula 2, formula 3, formula 4, formula 5, and formula 6. A formula of an index may comprise any five of formula 1, formula 2, formula 3, formula 4, formula 5, and formula 6.

In other cases, the exact make-up of a formula of an index may depend on the subjects, miRNAs, and/or statistical models used to generate the index.

Using the index to predict a disease outcome may generate at least two different populations of subjects, one having an index value higher than a threshold index value and one having an index value lower than the threshold index value. Using the index to predict a disease outcome may generate at least two different populations of subjects, one having an index value higher than or equal to the threshold index value and one having an index value lower than the threshold index value. In some cases, using the index to predict a disease outcome may generate at least two different populations of subjects, one having an index value higher than the threshold index value and one having an index value lower than or equal to the threshold index value. The two populations of subjects may have different disease outcomes. The different outcomes may comprise a difference of OS or PFS. The different outcomes may comprise a difference of outcome described here. Generating two different populations of subjects may refer to stratifying or stratification.

A threshold index value may comprise a mean, mode, median, maximum, minimum, range, first quartile, second quartile, third quartile, or fourth quartile value of the index values of a population of subjects. In some cases, a threshold index value may comprise a mean of index values of a population of subjects. In some cases, a threshold index value may comprise a mode of levels or of a population of subjects. In some cases, a threshold index value may comprise a median of levels or of a population of subjects. In some cases, a threshold index value may comprise a maximum of levels or of a population of subjects. In some cases, a threshold index value may comprise a minimum of levels or of a population of subjects. In some cases, a threshold index value may comprise a range of levels or of a population of subjects. In some cases, a threshold index value may comprise a first quartile of levels or of a population of subjects. In some cases, a threshold index value may comprise a second quartile of levels or of a population of subjects. In some cases, a threshold index value may comprise a third quartile of levels or of a population of subjects. In some cases, a threshold index value may comprise a fourth quartile value of levels or of a population of subjects.

In some cases, levels of miRNA can also be used to predict a disease outcome without being used to generate an index. For example, threshold levels of miRNAs can be used to predict a disease outcome. In some cases, using the level to predict a disease outcome may generate at least two different populations of subjects, one having a level of miRNA higher than the threshold level and one having a level of miRNA lower than the level. In some cases, using the threshold level of miRNA to predict the disease outcome may generate at least two different populations of subjects, one having a level of miRNA higher than or equal to the level and one having a level of miRNA lower than the threshold level. The two different populations of subjects may have different disease outcomes. In some cases, using the level to predict a disease outcome may generate at least two different populations of subjects, one having a level of miRNA higher than the threshold level and one having a level of miRNA lower than or equal to the level. The two populations of subjects may have different disease outcomes. The different outcomes may comprise a difference of OS or PFS. The different outcomes may comprise a difference of outcome described here. The levels of miRNA used to predict the disease outcome can comprise expression levels or normalized levels of miRNA. In some cases, the level(s) of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 210, 500, 1000, 2000, 5000, 10000 or more miRNAs can be used to predict the disease outcome without being used to generate an index. In some cases, the level(s) of at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 210, 500, 1000, 2000, 5000, or 10000 miRNAs can be used to predict the disease outcome without being used to generate an index.

In some instances, subjects can be stratified based on levels of miRNAs. In some instances, subjects can be stratified based on serum levels of miRNA. In some instances, the miRNA-stratified subjects can be analyzed with a Kaplan-Mayer Curve. In some instances, subjects can be stratified based on their generated index value. In some instances, the index-stratified subjects can be analyzed with a Kaplan-Meyer Curve. In some instances, the disease outcome analyzed with the Kaplan-Meyer curve can be Overall Survival (OS). In some instances, the disease outcome analyzed with the Kaplan-Mayer curve can be Progression-Free Survival (PFS).

In some cases, a population with an index value higher than the index may have a higher OS or PFS number than those of a population with an index value lower than the index. In some cases, a population with an index value higher than the index may have a lower OS or PFS number than those of a population with an index value lower than the index. In some cases, a population with an index value higher than the index may have a higher OS or PFS number than those of a population with an index value lower than the index. In some cases, a population with an index value higher than or equal to the index may have a lower OS or PFS number than those of a population with an index value lower than the index. In some cases, a population with an index value higher than or equal to the index may have a higher OS or PFS number than those of a population with an index value lower than the index. In some cases, a population with an index value higher than the index may have a lower OS or PFS number than those of a population with an index value lower than or equal to the index. In some cases, a population with an index value higher than the index may have a higher OS or PFS number than those of a population with an index value lower than or equal to the index.

In some cases, a population with an level of miRNA higher than the threshold level of the miRNA may have a higher OS or PFS number than those of a population with an level of miRNA lower than the threshold level of the miRNA. In some cases, a population with an level of miRNA higher than the threshold level of the miRNA may have a lower OS or PFS number than those of a population with an level of miRNA lower than the threshold level of the miRNA. In some cases, a population with an level of miRNA higher than the threshold level of the miRNA may have a higher OS or PFS number than those of a population with an level of miRNA lower than the threshold level of the miRNA. In some cases, a population with an level of miRNA higher than or equal to the threshold level of the miRNA may have a lower OS or PFS number than those of a population with an level of miRNA lower than the threshold level of the miRNA. In some cases, a population with an level of miRNA higher than or equal to the threshold level of the miRNA may have a higher OS or PFS number than those of a population with an level of miRNA lower than the threshold level of the miRNA. In some cases, a population with an level of miRNA higher than the threshold level of the miRNA may have a lower OS or PFS number than those of a population with an level of miRNA lower than or equal to the threshold level of the miRNA. In some cases, a population with an level of miRNA higher than the threshold level of the miRNA may have a higher OS or PFS number than those of a population with an level of miRNA lower than or equal to threshold level of the miRNA.

In some instances, a favorable or non-poor disease outcome may comprise a higher OS or PFS value. In some instances, a favorable or non-poor disease outcome may comprise a higher OS. In some instances, a favorable or non-poor disease outcome may comprise a higher PFS. In some instances, a favorable or non-poor disease outcome may comprise a higher OS and a higher PFS.

In some instances, a non-favorable or poor disease outcome may comprise a lower OS or PFS value. In some instances, a non-favorable or poor disease outcome may comprise a lower OS. In some instances, a non-favorable or poor disease outcome may comprise a lower PFS. In some instances, a non-favorable or poor disease outcome may comprise a lower OS and a lower PFS.

In some cases, a population with an index value higher than the index may have a lower OS number than those of a population with an index value lower than the index. In some cases, a population with an index value higher than the index may have a higher OS number than those of a population with an index value lower than the index. In some cases, a population with an index value higher than or equal to the index may have a lower OS number than those of a population with an index value lower than the index. In some cases, a population with an index value higher than or equal to the index may have a higher OS number than those of a population with an index value lower than the index. In some cases, a population with an index value higher than the index may have a lower OS number than those of a population with an index value lower than or equal to the index. In some cases, a population with an index value higher than the index may have a higher OS number than those of a population with an index value lower than or equal to the index.

In some cases, a population with an index value higher than the index may have a lower PFS number than those of a population with an index value lower than the index. In some cases, a population with an index value higher than the index may have a higher PFS number than those of a population with an index value lower than the index. In some cases, a population with an index value higher than or equal to the index may have a lower PFS number than those of a population with an index value lower than the index. In some cases, a population with an index value higher than or equal to the index may have a higher PFS number than those of a population with an index value lower than the index. In some cases, a population with an index value higher than the index may have a lower PFS number than those of a population with an index value lower than or equal to the index. In some cases, a population with an index value higher than the index may have a higher PFS number than those of a population with an index value lower than or equal to the index.

In some instances, the index can also be generated using an algorithm. In some instances, the index may be generated by a computer algorithm. In some instances, the algorithm may comprise a supervised learning algorithm, a semi-supervised learning algorithm, or a non-supervised learning algorithm. In some instances, the algorithm may comprise artificial neural network, Bayes classifier, blind source separation, decision tree, eigenmatrices, Gaussian radical basis function, joint-approximate diagonalization, kernel and polynomial kernel analysis, linear and nonlinear independent component analysis (ICA), natural gradient maximum likelihood estimation, non-Gaussianity analysis, principal component analysis (PCA), sequential floating forward selection, support vector machine, or a combination thereof.

### Subjects

In some instances, the index can be calculated using miRNA expression levels from between at least about 1 subject to about at least 10,000 subjects. In some instances, the number of subjects used to calculate the index can be at least about 1, 2, 3, 4, 5, 6, 7, 8, 9, 10, 11, 12, 13, 14, 15, 16, at about 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,050, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000, 3,050, 3,100, 3,150, 3,200, 3,250, 3,300, 3,350, 3,400, 3,450, 3,500, 3,550, 3,600, 3,650, 3,700, 3,750, 3,800, 3,850, 3,900, 3,950, 4,000, 4,050, 4,100, 4,150, 4,200, 4,250, 4,300, 4,350, 4,400, 4,450, 4,500, 4,550, 4,600, 4,650, 4,700, 4,750, 4,800, 4,850, 4,900, 4,950, 5,000, 5,050, 5,100, 5,150, 5,200, 5,250, 5,300, 5,350, 5,400, 5,450, 5,500, 5,550, 5,600, 5,650, 5,700, 5,750, 5,800, 5,850, 5,900, 5,950, 6,000, 6,050, 6,100, 6, 150, 6,200, 6,250, 6,300, 6,350, 6,400, 6,450, 6,500, 6,550, 6,600, 6,650, 6,700, 6,750, 6,800, 6,850, 6,900, 6,950, 7,000, 7,050, 7,100, 7,150, 7,200, 7,250, 7,300, 7,350, 7,400, 7,450, 7,500, 7,550, 7,600, 7,650, 7,700, 7,750, 7,800, 7,850, 7,900, 7,950, 8,000, 8,050, 8,100, 8,150, 8,200, 8,250, 8,300, 8,350, 8,400, 8,450, 8,500, 8,550, 8,600, 8,650, 8,700, 8,750, 8,800, 8,850, 8,900, 8,950, 9,000, 9,050, 9,100, 9,150, 9,200, 9,250, 9,300, 9,350, 9,400, 9,450, 9,500, 9,550, 9,600, 9,650, 9,700, 9,750, 9,800, 9,850, 9,900, 9,950, 10,000, or more.

In some instances, the index can be calculated using miRNA expression levels from between at most about 1 subject to about at most 10,000 subjects. In some instances, the number of subjects used to calculate the index can be at most about 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,050, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000, 3,050, 3,100, 3,150, 3,200, 3,250, 3,300, 3,350, 3,400, 3,450, 3,500, 3,550, 3,600, 3,650, 3,700, 3,750, 3,800, 3,850, 3,900, 3,950, 4,000, 4,050, 4,100, 4,150, 4,200, 4,250, 4,300, 4,350, 4,400, 4,450, 4,500, 4,550, 4,600, 4,650, 4,700, 4,750, 4,800, 4,850, 4,900, 4,950, 5,000, 5,050, 5,100, 5,150, 5,200, 5,250, 5,300, 5,350, 5,400, 5,450, 5,500, 5,550, 5,600, 5,650, 5,700, 5,750, 5,800, 5,850, 5,900, 5,950, 6,000, 6,050, 6,100, 6,150, 6,200, 6,250, 6,300, 6,350, 6,400, 6,450, 6,500, 6,550, 6,600, 6,650, 6,700, 6,750, 6,800, 6,850, 6,900, 6,950, 7,000, 7,050, 7,100, 7,150, 7,200, 7,250, 7,300, 7,350, 7,400, 7,450, 7,500, 7,550, 7,600, 7,650, 7,700, 7,750, 7,800, 7,850, 7,900, 7,950, 8,000, 8,050, 8,100, 8,150, 8,200, 8,250, 8,300, 8,350, 8,400, 8,450, 8,500, 8,550, 8,600, 8,650, 8,700, 8,750, 8,800, 8,850, 8,900, 8,950, 9,000, 9,050, 9,100, 9,150, 9,200, 9,250, 9,300, 9,350, 9,400, 9,450, 9,500, 9,550, 9,600, 9,650, 9,700, 9,750, 9,800, 9,850, 9,900, 9,950, or 10,000.

In some instances, the index can be calculated using miRNA expression levels from between about 1 subject to about 10,000 subjects. In some instances, the number of subjects used to calculate the index can 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,050, 2,100, 2,150, 2,200, 2,250, 2,300, 2,350, 2,400, 2,450, 2,500, 2,550, 2,600, 2,650, 2,700, 2,750, 2,800, 2,850, 2,900, 2,950, 3,000, 3,050, 3,100, 3,150, 3,200, 3,250, 3,300, 3,350, 3,400, 3,450, 3,500, 3,550, 3,600, 3,650, 3,700, 3,750, 3,800, 3,850, 3,900, 3,950, 4,000, 4,050, 4,100, 4,150, 4,200, 4,250, 4,300, 4,350, 4,400, 4,450, 4,500, 4,550, 4,600, 4,650, 4,700, 4,750, 4,800, 4,850, 4,900, 4,950, 5,000, 5,050, 5,100, 5,150, 5,200, 5,250, 5,300, 5,350, 5,400, 5,450, 5,500, 5,550, 5,600, 5,650, 5,700, 5,750, 5,800, 5,850, 5,900, 5,950, 6,000, 6,050, 6,100, 6, 150, 6,200, 6,250, 6,300, 6,350, 6,400, 6,450, 6,500, 6,550, 6,600, 6,650, 6,700, 6,750, 6,800, 6,850, 6,900, 6,950, 7,000, 7,050, 7,100, 7,150, 7,200, 7,250, 7,300, 7,350, 7,400, 7,450, 7,500, 7,550, 7,600, 7,650, 7,700, 7,750, 7,800, 7,850, 7,900, 7,950, 8,000, 8,050, 8,100, 8,150, 8,200, 8,250, 8,300, 8,350, 8,400, 8,450, 8,500, 8,550, 8,600, 8,650, 8,700, 8,750, 8,800, 8,850, 8,900, 8,950, 9,000, 9,050, 9,100, 9,150, 9,200, 9,250, 9,300, 9,350, 9,400, 9,450, 9,500, 9,550, 9,600, 9,650, 9,700, 9,750, 9,800, 9,850, 9,900, 9,950, or 10,000.

In some instances, the number of subjects used to calculate the index can be 442. In some instances, the number of subjects used to calculate the index can be at least about 442. In some instances, the number of subjects used to calculate the index can be at most about 442. In some instances, the numbers of subjects used to calculate the index can be 969. In some instances, the number of subjects used to calculate the index can be at least about 969. In some instances, the number of subjects used to calculate the index can be at most about 969. In some instances, the number of subjects used to calculate the index can be 180. In some instances, the number of subjects used to calculate the index can be at least about 180. In some instances, the number of subjects used to calculate the index can be at most about 180. In some instances, the number of subjects used to calculate the index can be 175. In some instances, the number of subjects used to calculate the index can be at least about 175. In some instances, the number of subjects used to calculate the index can be at most about 175. In some instances, the number of subjects used to calculate the index can be 68. In some instances, the number of subjects used to calculate the index can be at least about 68. In some instances, the number of subjects used to calculate the index can be at most about 68. In some instances, the number of subjects used to calculate the index can be 66. In some instances, the number of subjects used to generate the index can be at least about 66. In some instances, the number of subjects used to generate the index can be at most about 66.

In some instances, the subjects may have a specific cancer type or subtype. In some instances, the subjects used to generate the index may not have received any treatment. In some instances, the subjects may have a specific cancer type by selecting subjects with the same type of cancer. The subjects may have a first type or subtype of cancer but not a second type or subtype of cancer. In some instances, the first type or subtype of cancer or second type or subtype of cancer may comprise any type or subtype disclosed in the present disclosure. In some instances, the specific cancer type or subtype can be any of the cancer types or subtypes disclosed in the present disclosure. The subjects may have an ovarian cancer. The subjects may have endometrioid or mucinous ovarian cancer. The subjects may have endometrioid or serous ovarian cancer. The subjects may have mucinous or serous ovarian cancer. The subjects may have HGSOC. The subjects may have clear cell carcinoma. The subjects may have type I ovarian cancer. The subjects may have type II ovarian cancer.

In some cases, the subjects may have a stage of cancer. In some cases, the subjects may have a stage of cancer. In some cases, the subjects may have a stage 0 cancer. In some cases, the subjects may have a stage I cancer. In some cases, the subjects may have a stage IA cancer. In some cases, the subjects may have a stage IB cancer. In some cases, the subjects may have a stage IIA cancer. In some cases, the subjects may have a stage IIB cancer. In some cases, the subjects may have a stage IIIA cancer. In some cases, the subjects may have a stage IIIB cancer. In some cases, the subjects may have a stage IIIC cancer. In some cases, the subjects may have a stage IV cancer. In some cases, the subjects may have a stage of ovarian cancer. In some cases, the subjects may have a stage of ovarian cancer. In some cases, the subjects may have a stage 0 ovarian cancer. In some cases, the subjects may have a stage I ovarian cancer. In some cases, the subjects may have a stage IA ovarian cancer. In some cases, the subjects may have a stage IB ovarian cancer. In some cases, the subjects may have a stage IIA ovarian cancer. In some cases, the subjects may have a stage IIB ovarian cancer. In some cases, the subjects may have a stage IIIA ovarian cancer. In some cases, the subjects may have a stage IIIB ovarian cancer. In some cases, the subjects may have a stage IIIC ovarian cancer. In some cases, the subjects may have a stage IV ovarian cancer.

In some instances, subjects can be excluded from the index generation. In some instances, subjects with a different type of subtype of cancer can be excluded. In some instances, subjects with unclassifiable ovarian carcinoma samples can be excluded. In some instances, subjects with borderline malignancies can be excluded. In some instances, subjects with benign tumors can be excluded. In some instances, subjects can be excluded if the subject was treated prior to sample collection. Non-limiting examples of treatments prior to sample collection that can exclude subjects include: surgical operations, chemotherapy, or radiotherapy. In some instances, subjects can be excluded due to poor quality miRNA expression data from their sample. In some instances, subjects can be excluded due to poor quality microarray data. In some instances, specific miRNA molecules can be excluded from the index generation. In some instances, specific miRNA can be excluded due to low signal in the miRNA expression data.

miRNA Isolation and Identification

In some instances, the method can comprise extracting miRNAs from a sample. In some instances, the method can comprise extracting miRNA from a sample. In some instances, extracting RNA from a sample can comprise lysing the cell the RNA is located in. In some instances, RNA can be extracted from a sample using an organic extraction. In some instances, an organic extraction can use a phenol-Guanidine Isothiocyanate (GITC)-based solution. In some instances, RNA can be extracted from a sample using a silica-membrane based spin column. In some instances, RNA can be extracted from a sample using paramagnetic particles.

In some instances, miRNA can be extracted from a sample by contacting the sample with nanowires of the nanowire-incorporated devices. In some instances, extracting the miRNA can be performed under conditions in which the nanowires have a positive surface charge. For example, by contacting the nanowires with samples under pH conditions where the nanowires have a positive surface charge, free and EV-included forms of microRNA can be captured on the nanowires. In some instances, the sample fluid may be pH adjusted such that the nanowires have a positive surface charge. Alternatively, in some instances, the nanowires may be made of a material having a positive surface charge in the bodily fluid to match the pH of the sample.

In some instances, after the RNA is extracted, the RNA can be identified. In some instances, the RNA identified can be miRNA. In some instances, the miRNA can be identified using a miRNA microarray. In some instances, a microarray can comprise probes that can bind to a specific miRNA. In some instances, a microarray can comprise between about 100 to about 100,000 probes. In some instances, a microarray can comprise at least about 100, at least about 200, at least about 300, at least about 400, at least about 500, at least about 600, at least about 700, at least about 800, at least about 900, at least about 1,000, at least about 1,500, at least about 2,000, at least about 2,500, at least about 3,000, at least about 3,500, at least about 4,000, at least about 4,500, at least about 5,000, at least about 5,500, at least about 6,000, at least about 6,500, at least about 7,000, at least about 7,500, at least about 8,000, at least about 8,500, at least about 9,000, at least about 10,000, at least about 20,000, at least about 30,000, at least about 40,000, at least about 50,000, at least about 60,000, at least about 70,000, at least about 80,000, at least about 90,000, at least about 100,000, or more probes. In some instances, the microarray can comprise at least about 2.038 probes. In some instances, the microarray can comprise at most about 2.038. In some instances, the microarray can comprise 2,038. In some instances, the microarray can measure the expression level of a specific miRNA in a sample. In some instances, the microarray can measure the expression level of one or more specific RNAs simultaneously. In some instances, the microarray can measure the expression level of between about 100 to about 100,000 specific miRNAs simultaneously. In some instances, the microarray can measure the expression level of at least about 100, at least about 200, at least about 300, at least about 400, at least about 500, at least about 600, at least about 700, at least about 800, at least about 900, at least about 1,000, at least about 1,500, at least about 2,000, at least about 2,500, at least about 3,000, at least about 3,500, at least about 4,000, at least about 4,500, at least about 5,000, at least about 5,500, at least about 6,000, at least about 6,500, at least about 7,000, at least about 7,500, at least about 8,000, at least about 8,500, at least about 9,000, at least about 10,000, at least about 20,000, at least about 30,000, at least about 40,000, at least about 50,000, at least about 60,000, at least about 70,000, at least about 80,000, at least about 90,000, at least about 100,000, or more specific miRNAs simultaneously. In some instances, the microarray can measure the expression level of at least about 210 specific miRNAs simultaneously. In some instances, the microarray can measure the expression level of at most about 210 specific miRNAs simultaneously. In some instances, the microarray can measure the expression level of 210 specific miRNAs simultaneously.

In some instances, assaying a nucleic acid may comprise sequencing the nucleic acid or any derivative thereof. In other cases, assaying a nucleic acid may comprise identifying the nucleic acid or the derivative thereof. In some instances, assaying a nucleic acid may comprise identifying the sequence of the nucleic acid or the derivative thereof. In some instances, identifying the sequence of the nucleic acid or the derivative thereof may comprise identifying the mutation or variation of the nucleic acid or the derivative thereof, relative to the wildtype sequence of the nucleic acid. In some instances, assaying a nucleic acid may comprise identifying the modification of the nucleic acid sequence (e.g., an epigenetic modification). In some instances, assaying a nucleic acid may comprise identifying the sequence of the nucleic acid or the derivative thereof. In some instances, assaying a nucleic acid may comprise identifying the expression level of the nucleic acid or the derivative thereof. In some instances, assaying or identifying a nucleic acid may comprise sequencing the nucleic acid or a derivative thereof.

In some instances, sequencing may comprise whole genome sequencing. In some instances, sequencing may comprise whole genome methylation sequencing. In some instances, sequencing may comprise whole genome sequencing or whole genome methylation sequencing. In some instances, sequencing may comprise whole genome sequencing and whole genome methylation sequencing. In some instances, sequencing may comprise next-generation sequencing. In some instances, sequencing may comprise 2nd generation sequencing. In some instances, sequencing may comprise 3rd generation sequencing. In some instances, sequencing may comprise 4th generation sequencing.

In some instances, sequencing may comprise chain termination sequencing, high-throughput sequencing, mass spectrophotometry sequencing, massively parallel signature sequencing, Maxam-Gilbert sequencing, nanopore sequencing, primer walking, pyrosequencing, Sanger sequencing, semiconductor sequencing, sequencing-by-hybridization, sequencing-by-ligation, sequencing-by-synthesis, single-molecule sequencing, shotgun sequencing, bisulfite sequencing, or any combination thereof. In some instances, sequencing may comprise chain termination sequencing. In some instances, sequencing may comprise high-throughput sequencing. In some instances, sequencing may comprise mass spectrophotometry sequencing. In some instances, sequencing may comprise massively parallel signature sequencing. In some instances, sequencing may comprise Maxam-Gilbert sequencing. In some instances, sequencing may comprise nanopore sequencing. In some instances, sequencing may comprise primer walking. In some instances, sequencing may comprise pyrosequencing. In some instances, sequencing may comprise Sanger sequencing. In some instances, sequencing may comprise semiconductor sequencing. In some instances, sequencing may comprise sequencing-by-hybridization. In some instances, sequencing may comprise sequencing-by-ligation. In some instances, sequencing may comprise sequencing-by-synthesis. In some instances, sequencing may comprise single-molecule sequencing. In some instances, sequencing may comprise shotgun sequencing.

In some instances, identifying a nucleic acid may comprise sequencing, a PCR, a microarray analysis, or fluorescent hybridization. In some cases, identifying a nucleic acid may comprise a PCR. In some cases, identifying a nucleic acid may comprise a microarray analysis. In some instances, identifying a nucleic acid may comprise fluorescent hybridization. In some instances, a PCR may comprise an allele-specific PCR, an assembly PCR, an asymmetric PCR, a co-amplification at lower denaturation temperature-PCR, a dial-out PCR, a digital PCR, an emulsion PCR, a gene-specific PCR, a helicase-dependent PCR, a hot start PCR, an inverse PCR, a Klenow-based PCR, a ligation-mediated PCR, a methylation-specific PCR, a miniprimer PCR, a multiplex PCR, a nested PCR, a nested PCR, an overlap-extension PCR, a quantitative PCR, a real-time PCR, a thermal asymmetric interlaced PCR and touchdown PCR, a touchdown PCR, or a two-tailed PCR.

### Sample

In some instances, a sample may comprise an animal sample. In some instances, a sample may comprise mammalian sample. In some instances, a sample may comprise a primate sample. In some instances, a sample may comprise a human sample.

In some instances, a sample may be derived from a subject with a cancer. In some instances, a sample may be derived from a subject at risk of having or developing a cancer. In some instances, a sample may be derived from a subject without a cancer. In some instances, a sample may be derived from a subject that is healthy. In some instances, a sample may be derived from a subject without a risk of having or developing a cancer. In some instances, a sample may be derived from a subject that is healthy or without any disease conditions.

In some instances, a sample may comprise a cell-free sample. A cell-free sample may comprise a body sample that the cells have been removed. For example, a body sample may undergo centrifugation to remove any cells. In some cases, the cells may be filtered out from a body sample to form a cell-free sample. In other cases, the cells of a body sample may be digested or the plasma membranes of the cells are removed or destroyed to expose the components of the cells to form a cell-free sample. A cell-free sample may not comprise a cell. A cell-free sample may also be substantially free of cells. For example, a cell-free sample may comprise at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% of cells, relative to a body sample that cell-free sample is derived from. In some cases, a cell-free sample may be obtained non-invasively. In other cases, a cell-free sample may not be obtained invasively. In some cases, a non-invasive sampling procedure may not injure a subject in which a sample a obtained from. In other cases, an invasive sampling procedure may injure a subject in which a sample a obtained from. In some cases, an invasive sampling may obtain a sample that comprises at least a cell.

A sample can comprise a bodily fluid. In some instances, a sample may comprise a blood sample. In some instances, a sample may comprise a serum sample. In some instances, a sample may comprise a plasma sample. In some instances, a bodily fluid may comprise an intracellular bodily fluid or an extracellular bodily fluid. Non-limiting examples of extracellular bodily fluid can include intravascular fluid, interstitial fluid, lymphatic fluid, transcellular fluid, or a combination thereof. In some instances, a bodily fluid can also comprise ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. Bodily fluid may also comprise the blastocyl cavity, umbilical cord blood, or maternal circulation which may be of fetal or maternal origin. In some instances, the fluid sample is derived from a bodily fluid selected from among whole blood, sputum, serum, plasma, urine, cerebrospinal fluid, nipple aspirate, saliva, fine needle aspirate.

In some instances, a cell-free sample may not comprise a biopsy sample. Non-limiting examples of biopsy samples can include bone biopsy, a bone marrow biopsy, a breast biopsy, a gastrointestinal biopsy, a lung biopsy, a liver biopsy, a prostate biopsy, a nervous system biopsy, a urogenital biopsy, a lymph node biopsy, a muscle biopsy, a skin biopsy, a blood biopsy, a bodily fluid biopsy, a cardiac biopsy, an endometrial biopsy, an open biopsy, a sentinel lymph node biopsy, or any combinations thereof. In some instances, a biopsy can comprise a fine needle aspiration biopsy, a core needle biopsy, a vacuum-assisted biopsy, an excisional biopsy, a shave biopsy, a punch biopsy, an endoscopic biopsy, a laparoscopic biopsy, a bone marrow aspiration biopsy, a liquid biopsy, any derivatives herein and thereof, or any combinations herein and thereof. In some instances, a biopsy can comprise an incisional biopsy or an excisional biopsy.

### Extracellular Vesicles Exosomes

In some instances, the miRNA can be encapsulated in an extracellular vesicle. In some instances, the miRNA encapsulated in the extracellular vesicle can be delivered from one cell to a second cell. In some instances, the extracellular vesicle can comprise a micro-vesicle. In some instances, a micro-vesicle can be formed by direct outward budding/pinching of a cell's plasma membrane. In some instances, the extracellular vesicle can comprise an exosome. In some instances, an exosome can be enclosed within a single outer membrane which originates from the endosome. In some instances, the exosome can be secreted by all cell types. In some instances, the extracellular vesicle can be an apoptotic body. In some instances, an apoptotic body can be released by a dying cell.

In some cases, miRNAs used to predict a disease outcome or used to generate an index may be encompassed by exosomes originated from the cancer cell. In other cases, miRNAs used to predict a disease outcome or used to generate an index may be encompassed by exosomes originated from the non-cancer cell.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Preoperative Serum microRNAs as Potential Disease Outcome Prediction Markers in Ovarian Clear Cell Carcinoma

Provided herein are methods to identify miRNAs as markers to predict disease outcome in a subject. One example for the disease can comprise any ovarian cancer.

4,046 bodily fluid samples from healthy controls and subjects with ovarian tumors were investigated and their comprehensive microRNA profiles were generated using the 3D-Gene® miRNA Labeling kit and the 3D-Gene® Human miRNA Oligo Chip (Toray Industries, Inc.), which is described in Yokoi A et al. Integrated extracellular miRNA profiling for ovarian cancer screening. Nat Commun. 2018; 9:4319 and is hereby incorporated by reference in its entirety. The data is available through the NCBI database under the accession number GSE106817. In addition, to the 2,038 microRNAs in the datasets, 210 microRNAs were selected according to the criteria used in the previous study. Briefly, these microRNAs were detected in extracellular vesicles derived from ovarian cancer cells. TABLE 1 below lists the 210 identified miRNAs.

TABLE 1
Potential miRNA biomarkers for disease predictions
miRNA

hsa-miR-498
hsa-miR-451a
hsa-miR-4281
hsa-miR-4271
hsa-miR-6717-5p
hsa-miR-92a-3p
hsa-miR-486-3p
hsa-miR-3652
hsa-miR-4515
hsa-miR-4745-5p
hsa-miR-486-5p
hsa-miR-1249-3p
hsa-miR-4516
hsa-miR-4436b-5p
hsa-miR-5100
hsa-miR-17-3p
hsa-miR-3648
hsa-miR-4697-5p
hsa-miR-211-3p
hsa-miR-204-3p
hsa-miR-1290
hsa-miR-1202
hsa-miR-3141
hsa-miR-1246
hsa-miR-3135b
hsa-miR-5008-5p
hsa-miR-6722-3p
hsa-miR-1307-3p
hsa-miR-3622a-5p
hsa-miR-3180-3p
hsa-miR-3131
hsa-miR-4707-3p
hsa-miR-4505
hsa-miR-1181
hsa-miR-3197
hsa-miR-718
hsa-miR-4433a-3p
hsa-miR-4534
hsa-miR-4417
hsa-miR-5196-5p
hsa-miR-24-3p
hsa-miR-150-3p
hsa-miR-135a-3p
hsa-miR-1275
hsa-miR-1915-3p
hsa-miR-671-5p
hsa-miR-4298
hsa-miR-762
hsa-miR-4486
hsa-miR-6131
hsa-miR-4433a-5p
hsa-miR-6510-5p
hsa-miR-320a
hsa-miR-614
hsa-miR-3665
hsa-miR-4656
hsa-miR-4651
hsa-miR-4741
hsa-miR-1229-5p
hsa-miR-1227-5p
hsa-miR-1185-1-3p
hsa-miR-1247-3p
hsa-miR-5787
hsa-miR-5739
hsa-miR-373-5p
hsa-miR-663a
hsa-miR-1228-3p
hsa-miR-1228-5p
hsa-miR-642b-3p
hsa-miR-4665-3p
hsa-miR-365a-5p
hsa-miR-550a-5p
hsa-miR-1260b
hsa-miR-4728-5p
hsa-miR-4532
hsa-miR-4446-3p
hsa-miR-937-5p
hsa-miR-4632-5p
hsa-miR-887-3p
hsa-miR-320b
hsa-miR-1225-3p
hsa-miR-4443
hsa-miR-3162-5p
hsa-miR-4787-5p
hsa-miR-6124
hsa-miR-6090
hsa-miR-6075
hsa-miR-5001-5p
hsa-miR-4459
hsa-miR-642a-3p
hsa-miR-3620-5p
hsa-miR-3196
hsa-miR-4484
hsa-miR-4442
hsa-miR-4690-5p
hsa-miR-4463
hsa-miR-4674
hsa-miR-4730
hsa-miR-6087
hsa-miR-23b-3p
hsa-miR-3185
hsa-miR-625-3p
hsa-miR-3656
hsa-miR-4476
hsa-miR-1238-5p
hsa-miR-1237-5p
hsa-miR-711
hsa-miR-4270
hsa-miR-4466
hsa-miR-4710
hsa-miR-5195-3p
hsa-miR-5090
hsa-miR-23a-3p
hsa-miR-22-3p
hsa-miR-149-3p
hsa-miR-1914-3p
hsa-miR-1470
hsa-miR-1469
hsa-miR-4763-3p
hsa-miR-1207-5p
hsa-miR-187-5p
hsa-miR-1268a
hsa-miR-4667-5p
hsa-miR-4492
hsa-miR-4758-5p
hsa-miR-6716-5p
hsa-miR-4286
hsa-miR-4740-5p
hsa-miR-4488
hsa-miR-663b
hsa-miR-4294
hsa-miR-4327
hsa-miR-4513
hsa-miR-4688
hsa-miR-6724-5p
hsa-miR-6515-3p
hsa-miR-423-5p
hsa-miR-371a-5p
hsa-miR-4746-3p
hsa-miR-4726-5p
hsa-miR-197-5p
hsa-miR-2861
hsa-miR-3960
hsa-miR-3940-5p
hsa-miR-4447
hsa-miR-557
hsa-miR-3621
hsa-miR-2467-3p
hsa-miR-4430
hsa-miR-4739
hsa-miR-4429
hsa-miR-3937
hsa-miR-4454
hsa-miR-4707-5p
hsa-miR-6132
hsa-miR-6085
hsa-miR-6511a-5p
hsa-miR-744-5p
hsa-miR-4725-3p
hsa-miR-4449
hsa-miR-3158-5p
hsa-miR-1233-5p
hsa-miR-422a
hsa-miR-940
hsa-miR-939-5p
hsa-miR-760
hsa-miR-4750-5p
hsa-miR-4689
hsa-miR-4734
hsa-miR-4787-3p
hsa-miR-3154
hsa-miR-3917
hsa-miR-3679-3p
hsa-miR-3679-5p
hsa-miR-3663-3p
hsa-miR-4257
hsa-miR-4655-5p
hsa-miR-4640-5p
hsa-miR-1268b
hsa-miR-4634
hsa-miR-4783-3p
hsa-miR-1273g-3p
hsa-miR-638
hsa-miR-665
hsa-miR-296-5p
hsa-miR-4497
hsa-miR-4419b
hsa-miR-6126
hsa-miR-6125
hsa-miR-6076
hsa-miR-191-5p
hsa-miR-3188
hsa-miR-3195
hsa-miR-4687-3p
hsa-miR-4749-5p
hsa-miR-4530
hsa-miR-1185-2-3p
hsa-miR-4732-5p
hsa-miR-885-3p
hsa-miR-658
hsa-miR-1260a
hsa-miR-1225-5p
hsa-miR-4322
hsa-miR-4508
hsa-miR-4665-5p
hsa-miR-6089
hsa-miR-6088
hsa-miR-4695-5p
hsa-miR-652-5p
hsa-miR-1292-3p

Similar methods can also be used to identify other miRNAs for predicting other cancer outcomes in a subject. Other cancers can include any cancers described herein. The miRNAs can comprise miRNAs in a cell-free sample. The miRNAs can comprise serum miRNAs. miRNAs can also comprise any miRNAs in a bodily fluid described herein.

### Example 2: Extracellular microRNA Profiling for Disease Outcome Prediction in Subjects with High-Grade Serous Ovarian Carcinoma

Provided herein are methods to identify serum miRNAs as markers to predict HGSOC outcome in a subject.

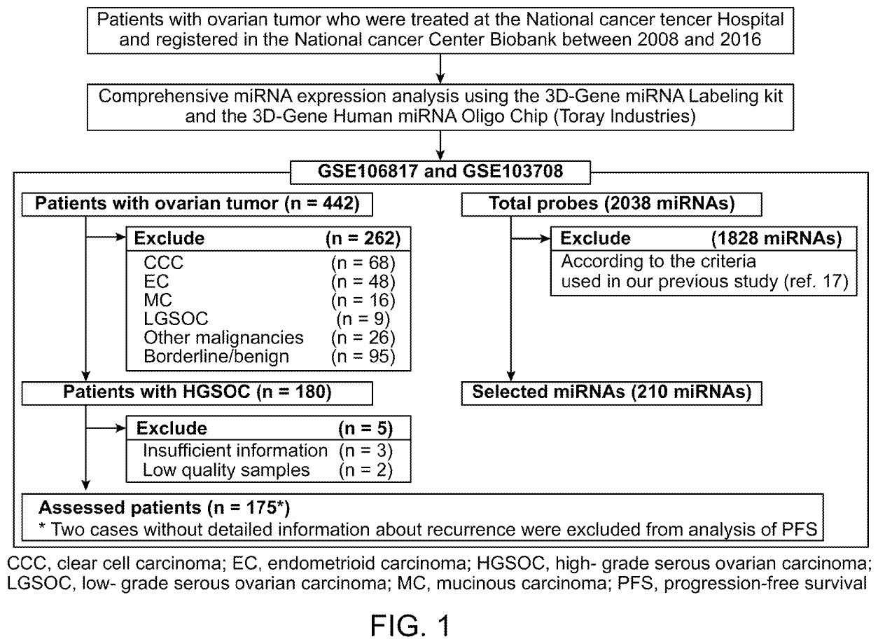

MicroRNA (miRNA) profiles for over 2000 microRNAs were generated from 4046 bodily fluid samples collected from control subjects and subjects with ovarian cancer using a 3D-Gene® miRNA Labeling kit and a 3D-Gene® Human miRNA Oligo Chip (Toray Industries, Inc.), and deposited to the NCBI database under the accession number GSE106817 described in Yokoi A et al. (described in EXAMPLE 1). The pretreatment serum miRNA profile of 442 additional subjects with ovarian tumors and 969 healthy controls were analyzed. Clinical information including age, disease stage, histological subtype, treatment status, and subject disease outcome were analyzed. The study was approved by the National Cancer Center Hospital Institutional Review Board (2015-376, 2016-29), and each participant provided written informed consent. 180 subjects with HGSOC were identified by excluding 262 subjects with other EOCs, other malignancies, borderline malignancies, or benign tumors FIG. 1. Then, after excluding three subjects with insufficient clinical information and two subjects with low-quality bodily fluid RNA samples, the association between bodily fluid samples miRNA profiles and the disease outcome prediction of 175 subjects with HGSOC was assessed. Among them, two subjects for whom details of recurrence were lost were excluded from the PFS analysis. In addition, of the 2038 miRNAs in the datasets, 210 miRNAs were selected according to the criteria used in the previous EXAMPLE 1. These miRNAs were detected in extracellular vesicles derived from ovarian cancer cells.

210 pretreatment serum miRNA levels from 175 patients with HGSOC were used to predict the disease outcome in the subjects. The characteristics of the 210 subjects (TABLE 2) with HGSOC included a median age of 60 years (range 28-82 years), and 92% of the subjects were diagnosed with International Federation of Gynecology and Obstetrics (FIGO) stage III or IV disease. Approximately half of the subjects received neoadjuvant chemotherapy, 151 subjects (86.3%) underwent complete or optimal cytoreductive surgery. Additionally, 150 subjects (85.7%) received adjuvant chemotherapy. The median follow-up period was 54.6 months (range, 3.5-144.1 months).

TABLE 2
Clinical characteristics of patients with
high-grade serous ovarian carcinoma

| Characteristic | Case (number of subjects (n) = 175) |
|---|---|
| Age (years) | Median (Range) |
| | 60 (28-32) |
| Stage | n |
| I | 4 (2.3) |
| II | 10 (5.7) |
| III | 104 (59.4) |
| IV | 57 (32.6) |
| Neoadjuvant chemotherapy | n |
| Yes | 99 (56.6) |
| No | 76 (43.4) |
| Surgery and residual tumor volume | n |
| Complete/optimal surgery | 151 (86.3) |
| Suboptimal surgery | 20 (11.4) |
| No surgery | 4 (2.3) |
| Adjuvant chemotherapy | n |
| Yes | 150 (85.7) |
| No | 25 (14.3) |

Note:
Data are shown as n (%) unless otherwise noted

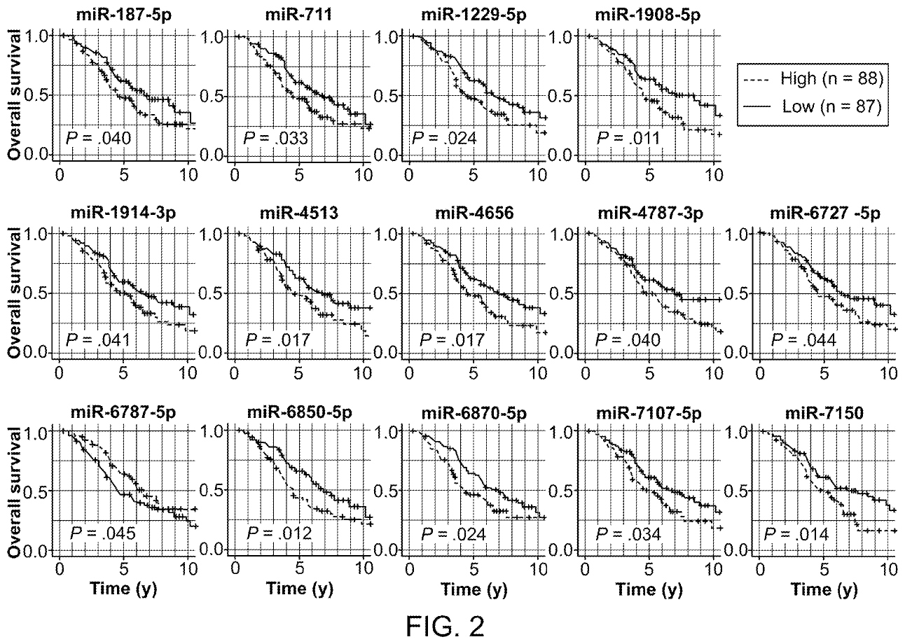

To investigate the association of the potential miRNAs (TABLE 1) on OS, Kaplan-Meier curves were generated after subjects were stratified into high and low expression groups based on the median level of each miRNA. Thirteen miRNAs were associated with significantly poorer OS (miR-187-5p, P=0.040; miR-711, P=0.033; miR-1229-5p, P=0.024; miR-1908-5p, P=0.011; miR-1914-3p, P=0.041; miR-4513, P=0.017; miR-4656, P=0.017; miR-4787-3p, P=0.040; miR-6727-5p, P=0.044; miR-6850-5p, P=0.012; miR-6870-5p, P=0.024; miR-7107-5p, P=0.034; miR-7150, P=0.014; FIG. 2). In contrast, miR-6787-5p was an miRNA associated with a favorable OS (P=0.045; FIG. 2). Cox regression analysis was performed for OS and PFS using the 14 miRNAs as a continuous variable. Univariate Cox regression analysis for OS was performed and hazard ratios (HRs) of miR-6870-5p and miR-187-5p were calculated at 1.383 (P=0.011) and 1.394 (P=0.046), respectively. Similarly, a univariate Cox regression analysis for PFS was performed and the HRs of miR-6870-5p and miR-187-5p were calculated at 1.280 (P=0.027) and 1.375 (P=0.029), respectively. In addition, the HRs for three more miRNAs were calculated (miR-6727-5p [HR 1.469; P=0.009], miR-6850-5p [HR 1.641; P=0.012], and miR-1908-5p [HR 1.489; P=0.043]). TABLE 3 summarizes the Hazard ratios (HRs) and 95% confidence intervals (Cls) for (A) overall survival (OS) and (B) progression-free survival (PFS) were calculated using the levels of miRNAs as a continuous variable.

TABLE 3
Univariate Cox regression analysis of survival
of patients with high-grade serous ovarian carcinoma
according to micro RNA (miRNA) levels

| | HR (95% CI) | P-value |
|---|---|---|
| miRNA (A) | | |
| miR-6870-5p | 1.383 (1.077-1.775) | 0.011 |
| miR-187-5p | 1.394 (1.006-1.931) | 0.046 |
| miR-1908-5p | 1.488 (0.950-2.330) | 0.082 |
| miR-7150 | 1.255 (0.890-1.768) | 0.195 |
| miR-4656 | 1.219 (0.900-1.650) | 0.201 |
| miR-7107-5p | 1.186 (0.901-1.560) | 0.224 |
| miR-6787-5p | 0.817 (0.591-1.132) | 0.225 |
| miR-1229-5p | 1.183 (0.884-1.584) | 0.258 |
| miR-6727-5p | 1.221 (0.862-1.728) | 0.261 |
| miR-6850-5p | 1.294 (0.819-2.043) | 0.269 |
| miR-1914-3p | 1.197 (0.852-1.680) | 0.3 |
| miR-4787-3p | 1.094 (0.845-1.417) | 0.495 |
| miR-711 | 1.087 (0.779-1.516) | 0.623 |
| miR-4513 | 1.023 (0.724-1.445) | 0.897 |
| miRNA (B) | | |
| miR-6727-5p | 1.469 (1.102-1.958) | 0.009 |
| miR-6850-5p | 1.641 (1.115-2.415) | 0.012 |
| miR-6870-5p | 1.280 (1.028-1.595) | 0.027 |
| miR-187-5p | 1.375 (1.032-1.831) | 0.029 |
| miR-1908-5p | 1.489 (1.013-2.188) | 0.043 |
| miR-7150 | 1.232 (0.920-1.648) | 0.161 |
| miR-7107-5p | 1.163 (0.919-1.473) | 0.209 |
| miR-6787-5p | 0.865 (0.660-1.133) | 0.292 |
| miR-1229-5p | 1.141 (0.889-1.466) | 0.3 |
| miR-711 | 1.111 (0.828-1.489) | 0.492 |
| miR-4656 | 1.081 (0.829-1.410) | 0.564 |
| miR-1914-3p | 1.055 (0.786-1.416) | 0.72 |
| miR-4787-3p | 0.977 (0.790-1.209) | 0.83 |

Hazard ratios (HRs) and 95% confidence intervals (CIs) for (A) overall survival (OS) and (B) progression-free survival (PFS) were calculated using the levels of miRNAs as a continuous variable.

10 circulating miRNAs (miR-320a, miR-665, miR-3184-5p, miR-6717-5p, miR-4459, miR-6076, miR-3195, miR-1275, miR-3185, and miR-4640-5p) were used for the prediction model for discrimination between ovarian cancer and healthy control samples (Yokoi A. et al.) However, none of them were significantly associated with the prognosis of patients with HGSOC in this study.

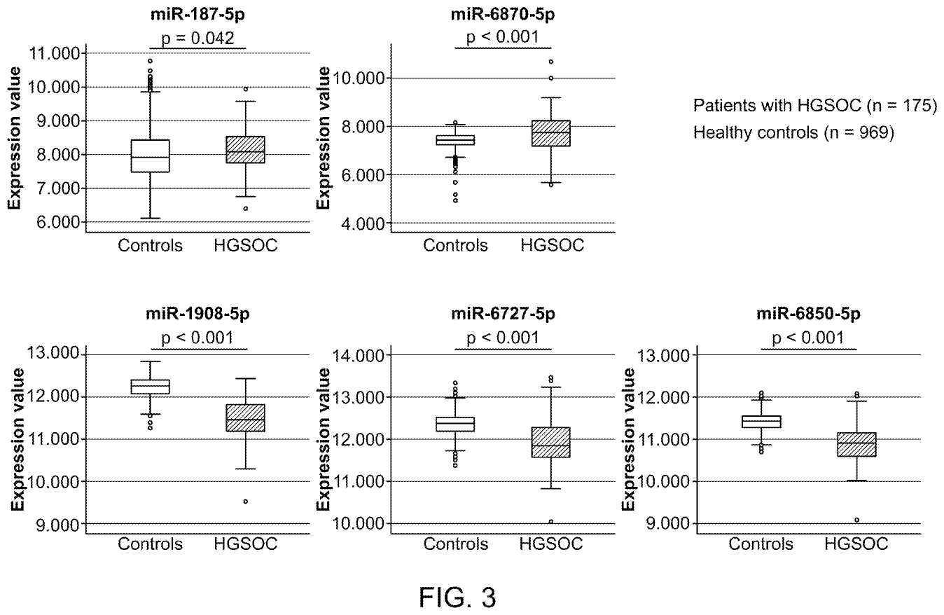

The levels of the disease outcome predictive miRNAs in healthy controls were evaluated, as shown in FIG. 3. miR-187-5p and miR-6870-5p were significantly higher in subjects with HGSOC than healthy controls (P=0.042 and P<0.001, respectively). The levels of miR-1908-5p, miR-6727-5p, and miR-6850-5p were significantly lower in HGSOC (P<0.001).

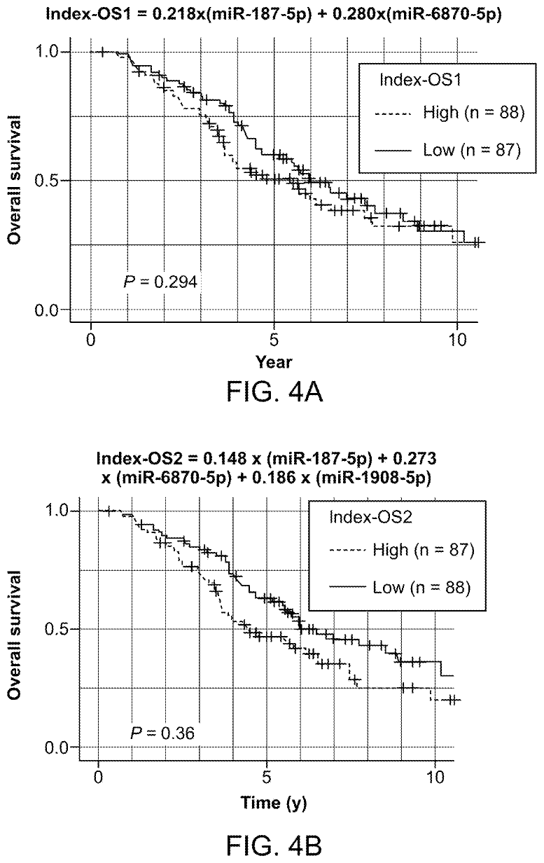

The index for OS was calculated using miR-187-5p, miR-6870-5p, and miR-1908-5p. First, index-OS1 was calculated using the expression values of two miRNAs (miR-187-5p and miR-6870-5p), but the Kaplan-Meier curves for OS showed no significant difference between the high and low groups (P=0.294; FIG. 4A). Thus, the index-OS2 was calculated using miR-187-5p, miR-6870-5p, and miR-1908-5p, which was the third OS-related miRNA with marginal significance (P=0.082; TABLE 3). The patient characteristics stratified by index-OS2 are shown in TABLE 4.

TABLE 4
Characteristics of patients with high-grade serous ovarian
carcinoma, stratified by index-OS2 = 0.148 ×
(miR-187-5p) + 0.273 × (miR-6870-5p) + 0.186 × (miR-1908-5p)

| Characteristic | Low (n = 87) | High (n = 88) | P value (P) |
|---|---|---|---|
| Age (Median year | 59 | 61.5 | 0.189 |
| (range)) | (34-82) | (28-82) | |
| Stage | | | |
| I | 3 (3.4) | 1(1.1) | 0.082 |
| II | 8 (9.2) | 2 (2.3) | |
| III | 51 (58.6) | 53 (60.2) | |
| IV | 25 (28.7) | 32 (36.4) | |
| Surgery and residual | | | |
| tumor volume | | | |
| Complete/optimal | 78 | 73 | 0.199 |
| surgery | (89.7) | (83.0) | |
| Suboptimal surgery/ | 9 | 15 | |
| No surgery | (10.3) | (17.0) | |
| miRNA levels | | | |
| (Median (range)) | | | |
| miR-187-5p | 7.836 | 8.297 | <0.001 |
| | (6.762-9.113) | (6.400-9.945) | |
| miR-6870-5p | 7.162 | 8.234 | <0.001 |
| | (5.543-8.084) | (7.362-10.688) | |
| miR-1908-5p | 11.237 | 11.691 | <0.001 |
| | (10.314-11.993) | (9.520-12.455) | |
| Index-OS2 | 5.237 | 5.658 | <0.001 |
| | (4.501-5.455) | (5.456-6.094) | |

Note:
Data are shown as n (%) unless otherwise noted.

The Kaplan-Meier curves for OS showed that patients with a high index had significantly shorter OS than those with a low index (P=0.036; FIG. 4B).

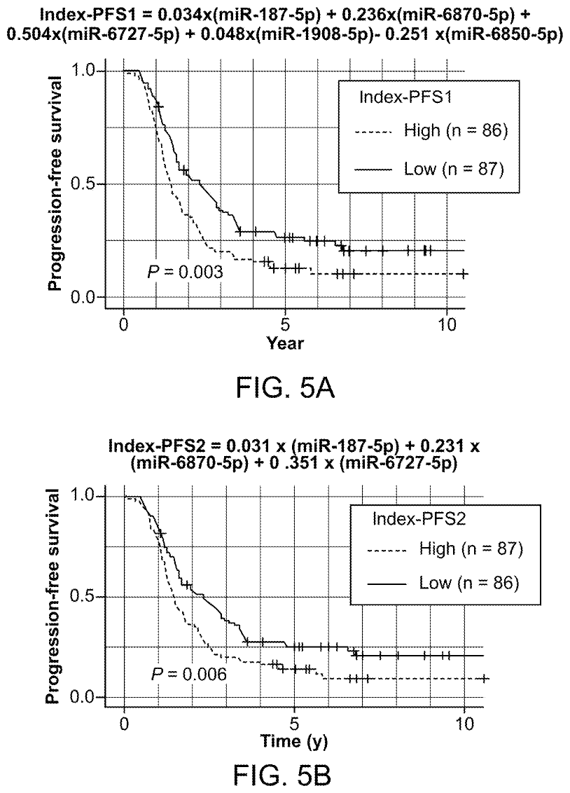

Similarly, the index for PFS was calculated using the five miRNAs (miR-187-5p, miR-6870-5p, miR-6727-5p, miR-1908-5p, and miR-6850-5p), and the Kaplan-Meier curves for PFS showed that patients with high index-PFS1 had significantly shorter PFS than those with low index-PFS1 (P=0.003; FIG. 5A). Index-PFS1 was then simplified, and index-PFS2 was created using miR-187-5p, miR-6870-5p, and miR-6727-5p. Patient characteristics stratified by index-PFS2 are shown in TABLE 5.

TABLE 5
Characteristics of patients with high-grade serous ovarian
carcinoma, stratified by index-PFS2 = 0.031 ×
(miR-187-5p) + 0.231 × (miR-6870-5p) + 0.351 × (miR-6727-5p)

| Characteristic | Low (n = 87) | High (n = 86) | P value |
|---|---|---|---|
| Age (Median year | 60 | 61 | 0.356 |
| (range)) | (34-82) | (38-82) | |
| Stage | | | |
| I | 2 (2.3) | 2 (2.3) | 0.032 |
| II | 8 (9.2) | 2 (2.3) | |
| III | 54 (62.1) | 48 (55.8) | |
| IV | 23 (26.4) | 34 (39.5) | |
| Surgery and residual | | | |
| tumor volume | | | |
| Complete/optimal | 77 | 73 | 0.484 |
| surgery | (88.5) | (84.9) | |
| Suboptimal | 10 | 13 | |
| surgery/No surgery | (11.5) | (15.1) | |
| miRNA levels | | | |
| (Median (range)) | | | |
| miR-187-5p | 7.819 | 8.348 | <0.001 |
| | (6.400-9.113) | (7.212-9.945) | |
| miR-6870-5p | 7.236 | 8.084 | <0.001 |
| | (5.543-10.688) | (6.606-10.039) | |
| miR-1908-5p | 11.664 | 12.262 | <0.001 |
| | (10.048-12.787) | (11.279-13.473) | |
| Index-PFS2 | 6.041 | 6.396 | <0.001 |
| | (5.553-6.204) | (6.205-6.983) | |

Note:
Data are shown as n (%) unless otherwise noted.

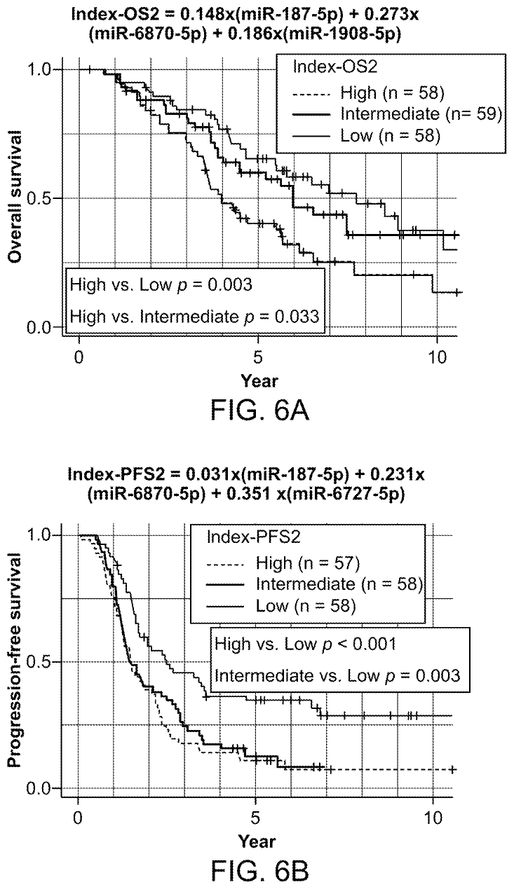

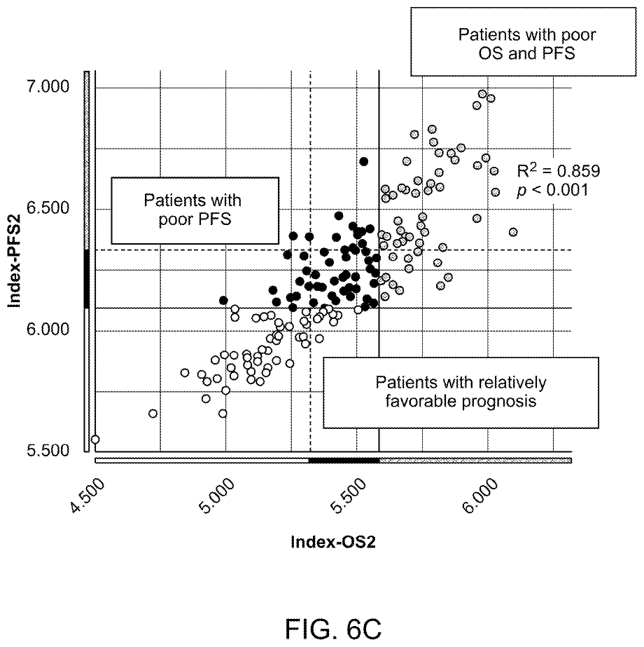

The Kaplan-Meier curves for PFS showed that patients with a high index had significantly shorter PFS than those with a low index (P=0.006; FIG. 5B). Therefore, index-PFS2 was sufficient to predict early recurrence. In addition, the patients were stratified into three groups (low, intermediate, and high) based on index-OS2 and index-PFS2. The Kaplan-Meier curves for OS showed that patients with a high index had significantly poorer OS than those with low and intermediate indices (P=0.003 and P=0.033, respectively; FIG. 6A). Moreover, the Kaplan-Meier curves for PFS showed that patients with a low index had significantly more favorable PFS than those with high and intermediate indices (P<0.001 and P=0.003, respectively; FIG. 6B). The correlation between index-OS2 and index-PFS2 was evaluated using the Pearson correlation coefficient, which revealed a significant correlation (R²=0.859, P<0.001; FIG. 6C). Therefore, patients with intermediate indices were considered to have relatively poor PFS but favorable OS.

The indices for OS (index-OS1 and index-OS2) and for PFS (index-PFS1 and index-PFS2) are summarized as follows:

$$1. Index - OS 1 = 0.218 \times ( miR - 187 - 5 p ) + 0.280 \times ( m i R - 6 \; 870 - 5 p ) ;$$

$$2. Index - OS 2 = 0.148 \times ( miR - 187 - 5 p ) + 0.273 \times ( m i R - 6 \; 870 - 5 p ) + 0.186 \times ( m i R - 1 \; 908 - 5 p ) ;$$

$$3. Index - PFS 1 = 0.034 \times ( m i R - 187 - 5 p ) + 0.236 \times ( miR - 6 \; 870 - 5 p ) + 0.5 \; 04 \times ( miR - 6727 - 5 p ) + 0.048 \times ( m i R - 1 \; 908 - 5 p ) - 0.251 \times ( m i R - 6 \; 870 - 5 p ) ; and$$

$$4. Index - PFS 2 = 0.031 \times ( m i R - 187 - 5 p ) + 0.231 \times ( m i R - 6 \; 870 - 5 p ) + 0.351 \times ( m i R - 6 \; 727 - 5 p ) .$$

To evaluate whether the indices were independent disease outcome prediction factors, A multivariate Cox regression analysis for OS and PFS was performed. The association between FIGO stage and residual tumor volume at the time of debulking surgery (complete or optimal surgery vs suboptimal surgery or no surgery) was calculated (P=0.012 and P<0.001, respectively; TABLE:

6).

TABLE 6
Univariate and multivariate Cox regression analysis for
overall survival (OS) and progression-free survival (PFS)
in patients with high-grade serous ovarian carcinoma

| Univariate | | Multivariate | |
|---|---|---|---|
| HR (95% CI) | P value | HR (95% CI) | P value |

<OS>

| | | | | |
|---|---|---|---|---|
| Age | 0.995 | 0.595 | 0.998 | 0.85 |
| | (0.975-1.015) | | (0.978-1.019) | |
| Stage (I, II, | 1.501 | 0.012 | 1.294 | 0.124 |
| III, or IV) | (1.095-2.057) | | (0.932-1.796) | |
| Residual tumor | 2.53 | 0 | 2.165 | 0.004 |
| volume^{a} | (1.527-4.190) | | (1.277-3.669) | |
| Index-OS2^{b} | 2.72 | 0.004 | 2.343 | 0.015 |
| | (1.382-5.352) | | (1.182-4.641) | |

<PFS>

| | | | | |
|---|---|---|---|---|
| Age | 1.009 | 0.268 | 1.013 | 0.119 |
| | (0.993-1.025) | | (0.997-1.029) | |
| Stage (I, II, | 1.532 | 0.001 | 1.39 | 0.014 |
| III, or IV) | (1.189-1.974) | | (1.069-1.809) | |
| Residual tumor | 1.913 | 0.008 | 1.771 | 0.024 |
| volume^{a} | (1.187-3.081) | | (1.077-2.914) | |
| Index-PFS2^{c} | 2.716 | 0.001 | 2.357 | 0.005 |
| | (1.521-4.851) | | (1.289-4.311) | |

^{a}0 for Complete/optimal surgery; 1 for Suboptimal surgery/no surgery.
^{b}Index-OS2 = 0.148 × (miR-187-5p) + 0.273 × (miR-6870-5p) + 0.186 × (miR-1908-5p).
^{c}Index-PFS2 = 0.031 × (miR-187-5p) + 0.231 × (miR-6870-5p) + 0.351 × (miR-6727-5p).

According to the univariate analysis, FIGO stage and residual tumor volume at the time of debulking surgery (complete or optimal surgery vs suboptimal surgery or no surgery) were also significantly associated with poor OS (P=0.012 and P<0.001, respectively; TABLE 6).

Multivariate analysis for OS showed that residual tumor volume and index-OS2 were independent poor disease outcome prediction factors (HR 2.165 [95% CI, 1.277-3.669], P=0.004; and HR 2.343 [95% CI, 1.182-4.641], P=0.015, respectively). Univariate analysis for PFS, FIGO stage and residual tumor volume at the time of debulking surgery were also associated with worse disease outcome (P=0.001 and P=0.008, respectively; TABLE 6). The multivariate analysis for PFS showed the association between FIGO stage and residual tumor volume (HR 1.390 [95% CI, 1.069-1.809], P=0.014; and HR 1.771 [95% CI, 1.077-2.914], P=0.024, respectively), showing that FIGO stage and residual tumor volume at the time of debulking surgery were also significantly associated with worse disease outcome. Moreover, index-PFS2 was also a significant independent poor disease outcome prediction factor (HR 2.357 [95% CI, 1.289-4.311], P=0.005; TABLE 6). Therefore, both indices were significant independent poor disease outcome prediction factors.

The functions of miR-187-5p, miR-6870-5p, and miR-1908-5p in EOC cells were investigated. A2780 and SK-OV-3 cell lines were purchased from the ATCC and maintained in RPMI-1640 (Nacalai Tesque) containing 10% FBS and antibiotics. The mirVana miRNA mimics for miR-187-5p (ID: MC12652), miR-1908-5p (ID: MC13846), miR-6870-5p (ID: MC27099), and negative control #1 were purchased from Thermo Fisher Scientific. Cells were seeded in 96-well plates and transfected with 20 nM mimic using Lipofectamine RNAi Max (Thermo Fisher Scientific). After 24, 48, and 72 h of incubation, cell viability was assessed using CellTiter-Glo 2.0 Cell Viability Assay (Promega) and a microplate reader (Gen5 Synergy H4; BioTek). For drug sensitivity analysis, after 24 h of transfection, the culture medium was replaced with cisplatin (Nichi-Iko Pharmaceutical) or docetaxel (Tokyo Chemical Industry) containing medium, and cells were incubated for 48 h. Cell viability was then assessed using CellTiter-Glo 2.0 Cell Viability Assay. To evaluate transfection efficacy, quantitative RT-PCR was carried out. Total RNA was extracted using the miRNeasy Mini Kit (Qiagen), and cDNA was synthesized using the TaqMan Advanced miRNA cDNA Synthesis Kit (Thermo Fisher Scientific). TaqMan Fast Advanced Master Mix (Thermo Fisher Scientific) and TaqMan Advanced miRNA Assays (assay IDs 479423_mir, 478735_mir, and 480864_mir; Thermo Fisher Scientific) were used for quantitative RT-PCR.

The Pearson correlation coefficient was used to evaluate the correlation between the two indices. The expression of miRNAs and cell viability was compared using Welch's t test. Differences at P<0.05 were considered as statistically significant.

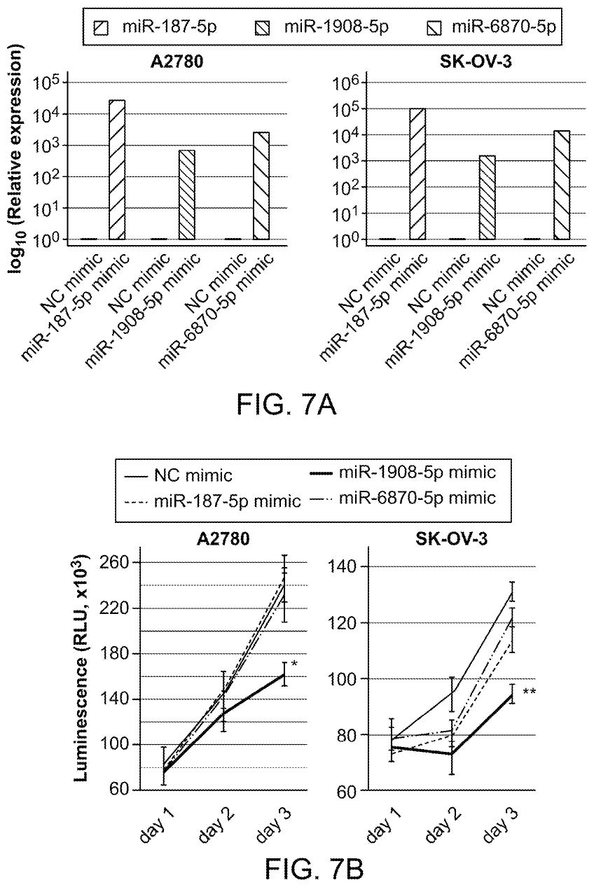

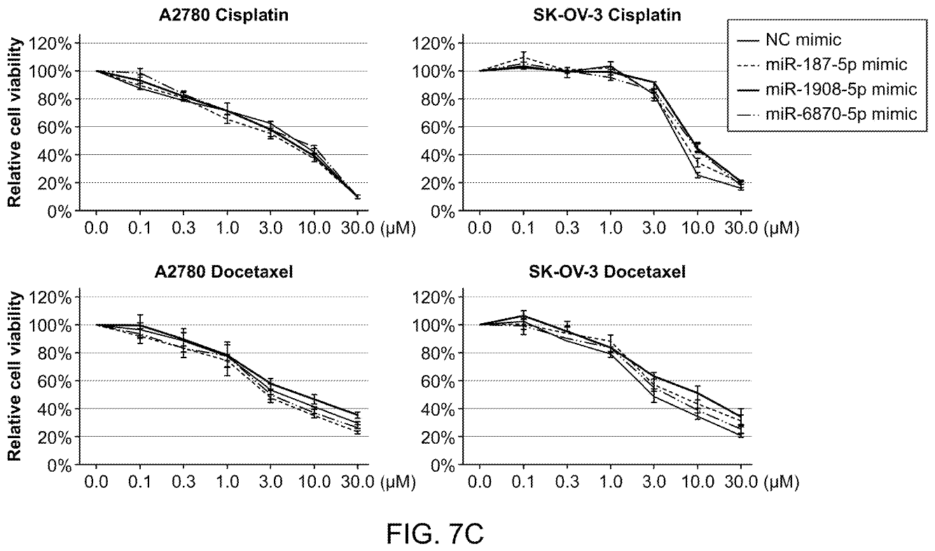

miR-187-5p, miR-6870-5p, and miR-1908-5p were each transfected in A2780 and SK-OV-3 cell lines (FIG. 7A). Proliferation assay revealed that miR-1908-5p significantly re-duced cell viability in both cell lines (A2780, P<. 05; and SK-OV-3, P<01; FIG. 7B). The remaining two miRNAs slightly inhibited cell proliferation in SK-OV-3 cells. We then evaluated the impact of the miRNAs on treatment resistance (FIG. 7C).

### Example 3: Extracellular microRNA Profiling for Disease Outcome Prediction in Subjects with Ovarian Clear Cell Carcinoma

Provided herein are methods to identify serum miRNAs as markers to predict ovarian clear cell carcinoma outcome in a subject.

210 microRNAs were selected according EXAMPLE 1. Twenty-five subjects with ovarian clear cell carcinoma and their formalin-fixed, paraffin-embedded samples (cases 1-20) and fresh-frozen surgical samples (cases 21-25) were used for the analysis. These samples were described in Yoshida K, Yokoi A, Sugiyama M, et al. Expression of the chrXq27.3 miRNA cluster in recurrent ovarian clear cell carcinoma and its impact on cisplatin resistance. Oncogene. 2021; 40:1255-1268, which is hereby incorporated by reference in its entirety.

Total RNA was extracted from formalin-fixed, paraffin-embedded samples using an miRNeasy FFPE Kit (Qiagen, Hilden, Germany) and fresh-frozen surgical samples using an miRNeasy Mini Kit (Qiagen). Comprehensive miRNA sequencing was performed according to the method described in Yoshida K, Yokoi A, Kagawa T, et al. Unique miRNA profiling of squamous cell carcinoma arising from ovarian mature teratoma: comprehensive miRNA sequence analysis of its molecular background. Carcinogenesis. 2019; 40:1435-1444) and is hereby incorporated by reference in its entirety.

442 subjects were selected with ovarian tumor who had the preoperative serum miRNA profile described in Yokoi A, Matsuzaki J, Yamamoto Y, et al. Integrated extracellular microRNA profiling for ovarian cancer screening. Nat Commun. 2018; 9:4319, which is hereby incorporated by reference in its entirety. In addition, the medical records, such as age, stage, histological subtype, residual tumor volume, adjuvant therapy, and recurrence or death events were retrospectively reviewed. The study was approved by the Ethics Committee of National Cancer Center Hospital Institutional (2015-376, 2016-29) and Nagoya University (2015-0237, 2017-0053, and 2017-0497), and each participant provided written informed consent.

Statistical analyses were performed with SPSS version 28 (IBM Corp., Armonk, NY). Overall survival (OS) was defined as the time from treatment initiation to death from any cause, while PFS was defined as the time from treatment initiation to tumor progression. Kaplan-Meier curves were used for the analysis of OS and PFS and were compared using the log-rank test. Univariate and multivariate Cox regression analyses were used to calculate hazard ratios (HRs) and 95% confidence intervals (CIs). The disease outcome prediction indices for OS and PFS were separately calculated based on a multivariate Cox regression model for miRNA candidates. The Pearson correlation coefficient was used to evaluate the correlation between the two indices.

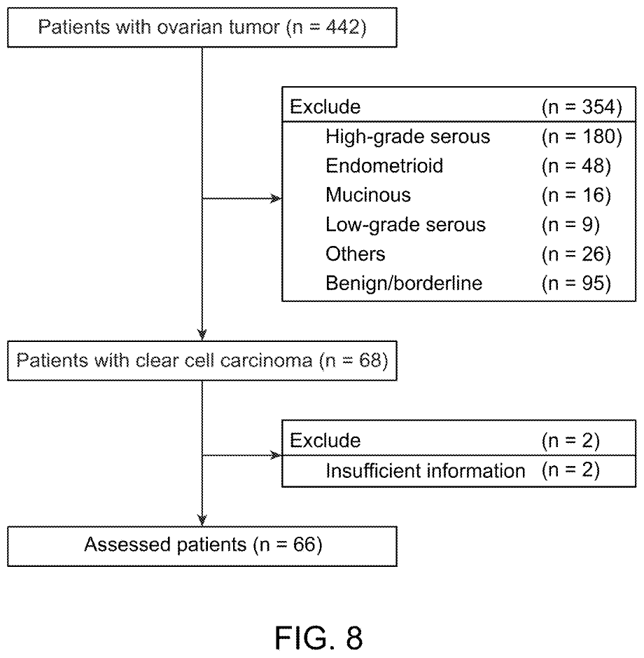

68 subjects with ovarian clear cell carcinoma were identified by excluding 354 subjects with other epithelial ovarian cancers, other malignancies, borderline malignancies, or benign tumors FIG. 8. After excluding two subjects with insufficient clinical information, the association between bodily fluid microRNA profiles and the disease outcome prediction of 66 subjects with ovarian clear cell carcinoma were assessed. The characteristics of these subjects are shown in TABLE 7.

TABLE 7
Characteristics of subjects with ovarian clear cell carcinoma

| Characteristic | Case (n = 66) |
|---|---|
| Age (Median year (range)) | 55.5(27-76) |
| FIGO stage | |
| I | 36 (54.5) |
| II | 9 (13.6) |
| III | 18 (27.4) |
| IV | 3 (4.5) |
| Cytoreductive surgery | |
| Complete | 65 (98.5) |
| Optimal | 1 (1.5) |
| Adjuvant chemotherapy | |
| Yes | 60 (90.9) |
| No | 6 (9.1) |

The median age of the subjects was 55.5 (range 27-76 years), and 36 subjects (54.5%) were diagnosed with International Federation of Gynecology and Obstetrics (FIGO) stage I disease. All the subjects except one received complete cytoreductive surgery. Additionally, 60 subjects (90.9%) received adjuvant chemotherapy, which was typically carboplatin plus paclitaxel combination chemotherapy. The median follow-up period was 64.3 months (range 8.0-153.3 months).

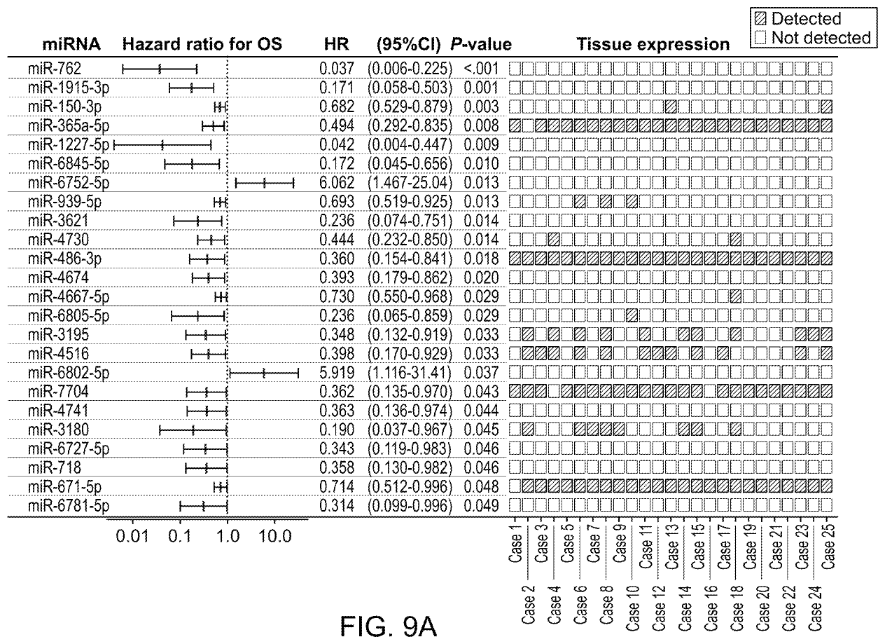

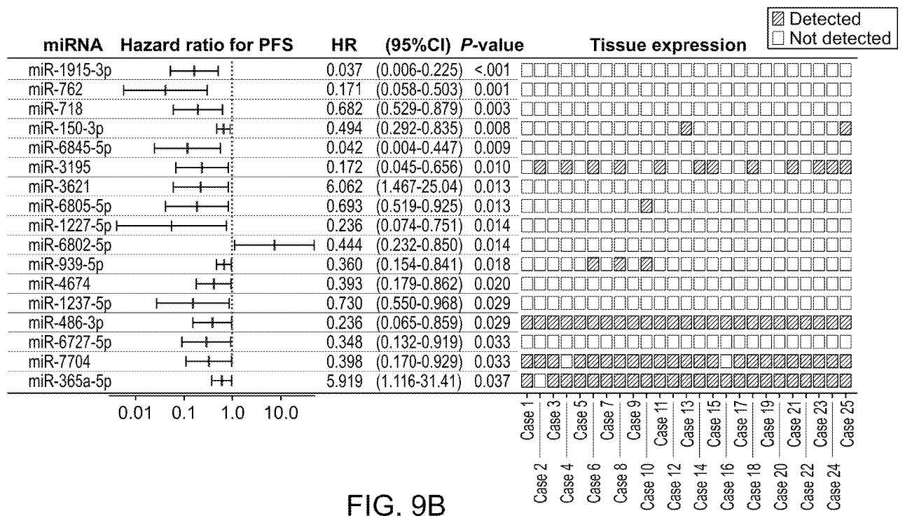

To investigate the disease outcome prediction impact of miRNAs on OS and PFS, a Cox regression analysis for OS and PFS was performed, using the 210 miRNAs as a continuous variable. Univariate Cox regression analysis for OS revealed that only two miRNAs were significantly associated with shorter OS and that 22 miRNAs were significantly associated with longer OS (FIG. 9A). Similarly, a univariate Cox regression analysis for PFS revealed that only one miRNA was significantly associated with shorter PFS and that 16 miRNAs were significantly associated with longer PFS (FIG. 9B). The expression of each miRNA derived from ovarian clear cell carcinoma tissue was assessed (FIGS. 9A-B). Twelve miRNAs were associated with OS and detected in ovarian clear cell carcinoma tissue, while the other twelve miRNAs were not detected in tissue (FIG. 9A). Similarly, seven miRNAs were associated with PFS and detected in ovarian clear cell carcinoma tissue, while the other ten miRNAs were not detected in tissue (FIG. 9B).

To evaluate whether the miRNAs were independent disease outcome prediction factors, a multivariate Cox regression analysis for OS and PFS was performed with each miRNA, age, stage, and residual tumor volume. According to the multivariate analysis, six of twelve miRNAs were associated with significantly favorable OS (miR-150-3p, hazard ratio [HR] 0.682, p=0.007; miR-3195, HR 0.365, p=0.017; miR-7704, HR 0.246, p=0.028; miR-365a-5p, HR 0.454, p=0.023; miR-4730, HR 0.358, p=0.015; miR-671-5p, HR 0.578, p=0.005, TABLE 8). Similarly, four of seven miRNAs were associated with significantly favorable PFS (miR-150-3p, HR 0.731, p=0.018; miR-3195, HR 0.283, p=0.003; miR-7704, HR 0.278, p=0.020; miR-6805-5p, HR 0.167, p=0.011), listed in TABLE 8 below.

TABLE 8
Summary of multivariate Cox regression analysis
for OS and PFS and subjects' characteristics

| | |
|---|---|
| HR (95% CI) | P value |

<OS>

| | | |
|---|---|---|
| Age | 0.982 (0.931-1.035) | 0.492 |
| Stage (I, II, III, or IV) | 2.371 (1.354-4.121) | 0.002 |
| Residual tumor volume | 2.989 (0.367-24.37) | 0.306 |
| miR-150-3p | 0.682 (0.516-0.901) | 0.007 |

<PFS>

| | | |
|---|---|---|
| Age | 0.992 (0.943-1.043) | 0.751 |
| Stage (I, II, III, or IV) | 2.591 (1.573-4.269) | <0.001 |
| Residual tumor volume | 2.223 (0.282-17.53) | 0.448 |
| miR-150-3p | 0.731 (0.564-0.948) | 0.018 |

<OS>

| | | |
|---|---|---|
| Age | 1.004 (0.951-1.059) | 0.894 |
| Stage (I, II, III, or IV) | 2.498 (1.405-4.442) | 0.002 |
| Residual tumor volume | 3.292 (0.393-27.58) | 0.272 |
| miR-3195 | 0.365 (0.160-0.832) | 0.017 |

<PFS>

| | | |
|---|---|---|
| Age | 1.015 (0.966-1.067) | 0.553 |
| Stage (I, II, III, or IV) | 2.898 (1.680-4.998) | <0.001 |
| Residual tumor volume | 2.677 (0.332-21.36) | 0.355 |
| miR-3195 | 0.283 (0.122-0.659) | 0.003 |

<OS>

| | | |
|---|---|---|
| Age | 1.006 (0.955-1.059) | 0.834 |
| Stage (I, II, III, or IV) | 2.390 (1.407-4.061) | 0.001 |
| Residual tumor volume | 4.303 (0.476-38.88) | 0.194 |
| miR-7704 | 0.246 (0.071-0.856) | 0.028 |

<PFS>

| | | |
|---|---|---|
| Age | 1.009 (0.962-1.058) | 0.706 |
| Stage (I, II, III, or IV) | 2.672 (1.634-4.368) | <0.001 |
| Residual tumor volume | 3.193 (0.374-27.25) | 0.288 |
| miR-7704 | 0.278 (0.094-0.821) | 0.02 |

<OS>

| | | |
|---|---|---|
| Age | 0.996 (0.945-1.049) | 0.866 |
| Stage (I, II, III, or IV) | 2.364 (1.410-3.962) | 0.001 |
| Residual tumor volume | 3.238 (0.395-26.55) | 0.274 |
| miR-365a-5p | 0.454 (0.230-0.897) | 0.023 |

<PFS>

| | | |
|---|---|---|
| Age | 1.011 (0.963-1.062) | 0.661 |
| Stage (I, II, III, or IV) | 2.412 (1.485-3.919) | <0.001 |
| Residual tumor volume | 2.057 (0.262-16.18) | 0.493 |
| miR-365a-5p | 0.789 (0.554-1.126) | 0.192 |

<OS>

| | | |
|---|---|---|
| Age | 0.995 (0.941-1.051) | 0.848 |
| Stage (I, II, III, or IV) | 2.507 (1.429-4.396) | 0.001 |
| Residual tumor volume | 2.397 (0.300-19.13) | 0.409 |
| miR-4730 | 0.358 (0.157-0.817) | 0.015 |

<PFS>

| | | |
|---|---|---|
| Age | 1.003 (0.952-1.055) | 0.92 |
| Stage (I, II, III, or IV) | 2.637 (1.598-4.351) | <0.001 |
| Residual tumor volume | 1.756 (0.228-13.54) | 0.589 |
| miR-4730 | 0.502 (0.235-1.071) | 0.075 |

<OS>

| | | |
|---|---|---|
| Age | 1.004 (0.949-1.062 | 0.881 |
| Stage (I, II, III, or IV) | 2.618 (1.535-4.463) | <0.001 |
| Residual tumor volume | 2.398 (0.305-18.83) | 0.406 |
| miR-671-5p | 0.578 (0.395-0.847) | 0.005 |

<PFS>

| | | |
|---|---|---|
| Age | 1.009 (0.959-1.061) | 0.725 |
| Stage (I, II, III, or IV) | 2.639 (1.627-4.282) | <0.001 |
| Residual tumor volume | 1.893 (0.245-14.60) | 0.54 |
| miR-671-5p | 0.708 (0.463-1.083) | 0.111 |

<OS>

| | | |
|---|---|---|
| Age | 1.027 (0.973-1.085) | 0.333 |
| Stage (I, II, III, or IV) | 2.515 (1.388-4.557) | 0.002 |
| Residual tumor volume | 3.360 (0.395-28.57) | 0.267 |
| miR-6805-5p | 0.444 (0.128-1.537) | 0.2 |

<PFS>

| | | |
|---|---|---|
| Age | 1.028 (0.979-1.079) | 0.265 |
| Stage (I, II, III, or IV) | 2.828 (1.620-4.937) | <0.001 |
| Residual tumor volume | 2.607 (0.318-21.35) | 0.372 |
| miR-6805-5p | 0.167 (0.043-0.659) | 0.011 |

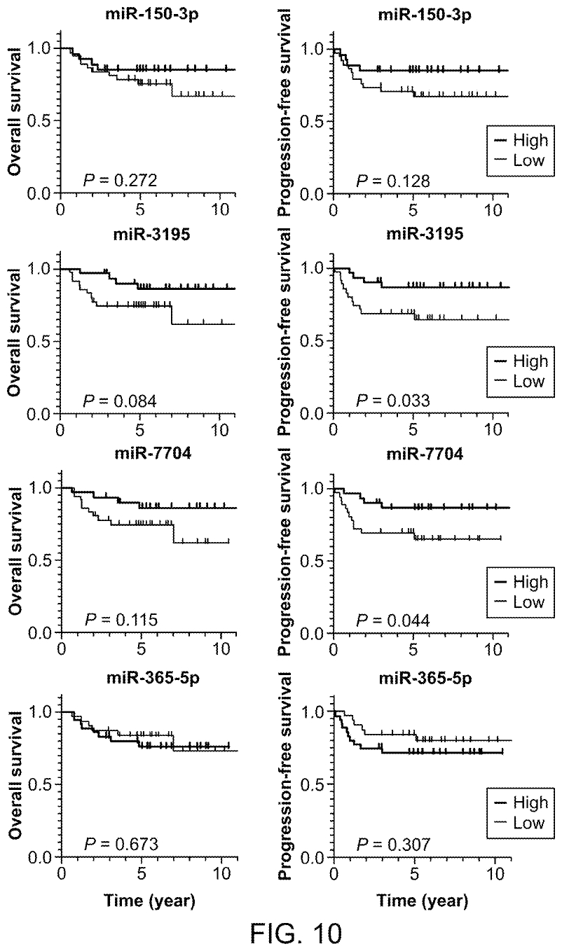

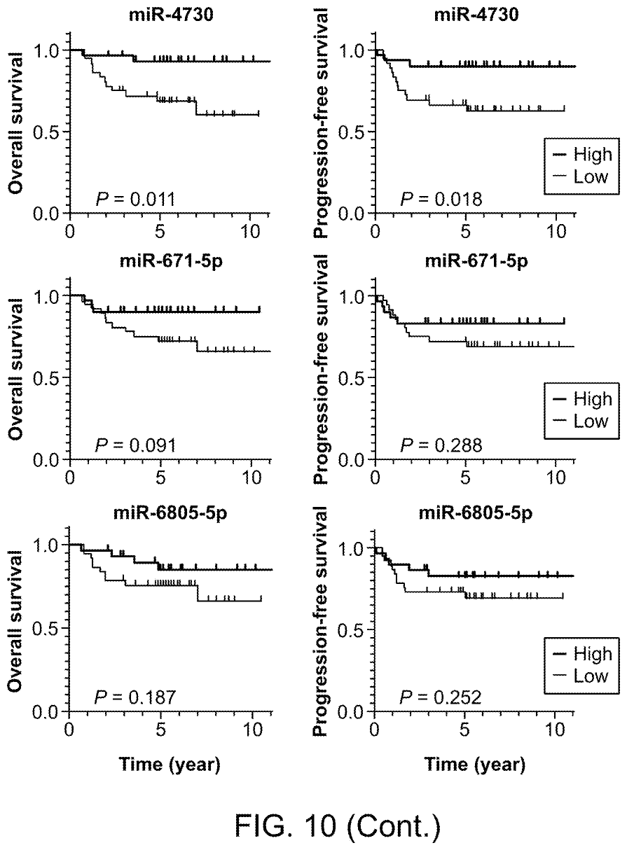

Kaplan-Meier curves were generated after patients were stratified into high and low expression groups based on the median level of seven miRNAs (miR-150-3p, miR-3195, miR-7704, miR-365a-5p, miR-4730, and miR-671-5p). Of six miRNAs (miR-150-3p, miR-3195, miR-7704, and miR-6805-5p), only one miRNA was associated with significantly favorable OS [miR-4730 (p=0.011), as shown in FIG. 10. Of four miRNAs (miR-150-3p, miR-3195, miR-7704, miR-365a-5p, miR-4730, miR-671-5p, and miR-6805-5p), two miRNAs were associated with significantly favorable PFS [miR-3195 (p=0.033); miR-7704 (p=0.044).

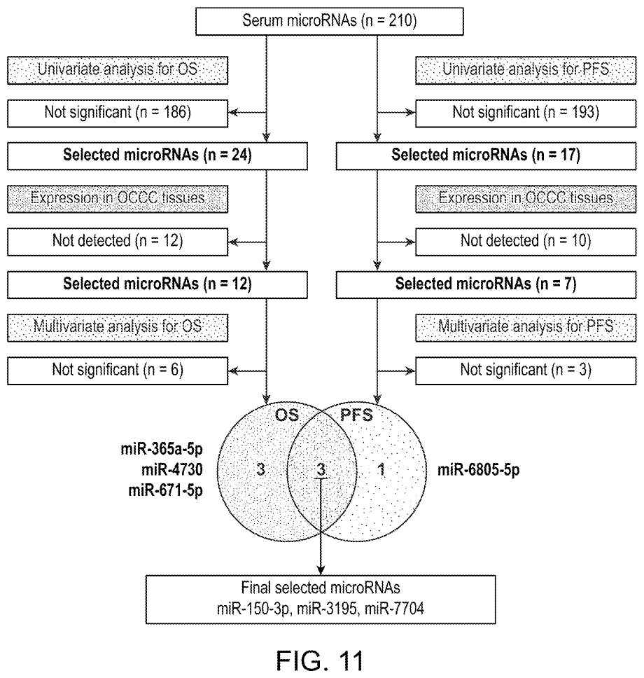

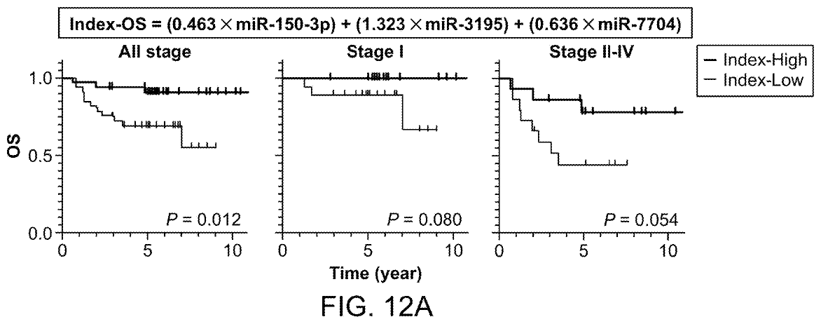

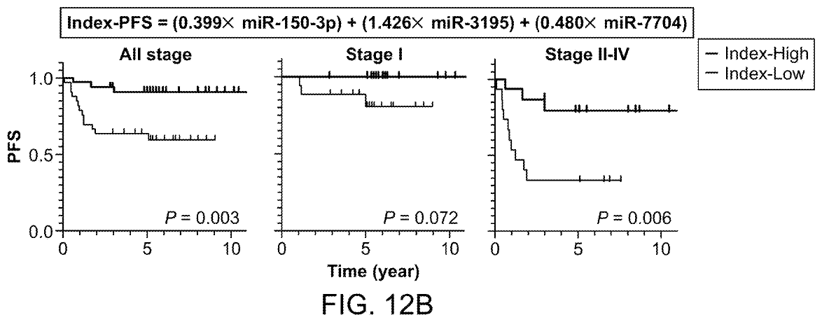

To increase the accuracy of the disease outcome prediction using these miRNAs, the index for OS was calculated. First, index-OS was calculated using the bodily fluid expression values of three miRNAs (miR-150-3p, miR-3195, and miR-7704) that were common candidate in both OS and PFS Cox regression analysis, as shown in FIG. 11. The index for OS (index-OS) and was as follows: “(index-OS)=(0.463×miR-150-3p)+ (1.323×miR-3195)+ (0.636×miR-7704).” The Kaplan-Meier curves for OS showed that patients with a low index significantly shorter OS than those with a high index (p=0.012), as shown in FIG. 12A. Similarly, the index for PFS was calculated using the same three miRNAs (miR-150-3p, miR-3195, and miR-7704). The index for PFS (index-PFS) was as follows: “(index-PFS)=(0.399×[miR-150-3p])+ (1.426×[miR-3195])+ (0.480×[miR-7704].” The Kaplan-Meier curves for PFS showed that patients with low index-PFS had significantly shorter PFS than those with high index-PFS (p=0.003), as shown in FIG. 12B). Therefore, indices-OS and -PFS were enough to predict disease outcome prediction and early recurrence. The patient characteristics stratified by the indices-OS and -PFS are shown in TABLE 9.

TABLE 9
Patient characteristics stratified by the indices-OS and -PFS

| | Index-OS | | | | | Index-PFS | | | | |
|---|---|---|---|---|---|---|---|---|---|---|
| | High(n = | | Low(n = | | | High(n = | | Low(n = | | |
| Characteristic | 33) | | 33) | | P-value | 33) | | 33) | | P-value |
| Age (Median | 55 | (27-73) | 55.5 | (34-75) | 0.379 | 55 | (27-73) | 57 | (34-76) | 0.17 |
| year (range)) | | | | | | | | | | |
| FIGO stage | | | | | | | | | | |
| I | 18 | (54.5) | 18 | (54.5) | 0.806 | 18 | (54.5) | 18 | (54.5) | 0.806 |
| II | 5 | (15.2) | 4 | (12.1) | | 5 | (15.2) | 4 | (12.1) | |
| III | 7 | (21.2) | 11 | (33.3) | | 7 | (21.2) | 11 | (33.3) | |
| IV | 3 | (9.1) | 0 | (0) | | 3 | (9.1) | 0 | (0) | |
| Cytoreductive | | | | | | | | | | |
| surgery | | | | | | | | | | |
| Complete | 32 | (97.0) | 33 | (100) | 0.321 | 32 | (97.0) | 33 | (100) | 0.321 |
| Optimal | 1 | (3.0) | 0 | (0) | | 1 | (3.0) | 0 | (0) | |
| Adjuvant | | | | | | | | | | |
| chemotherapy | | | | | | | | | | |
| Yes | 29 | (87.9) | 31 | (93.9) | 0.4 | 29 | (87.9) | 31 | (93.9) | 0.4 |
| No | 4 | (12.2) | 2 | (6.1) | | 4 | (12.2) | 2 | (6.1) | |
| microRNA | | | | | | | | | | |
| levels | | | | | | | | | | |
| Median | | | | | | | | | | |
| (range) | | | | | | | | | | |
| miR-150-3p | 6.216 (4.8558- | | 5.547 (0.082- | | <0.001 | 6.192 (4.8558- | | 5.817 (0.082- | | <0.001 |
| | 7.898) | | 6.918) | | | 7.898) | | 7.070) | | |
| miR-3195 | 8.757 (8.040- | | 8.377 (6.784- | | <0.001 | 8.757 (8.212- | | 8.569 (6.784- | | <0.001 |
| | 9.232) | | 8.890) | | | 9.232) | | 8.890) | | |
| miR-7704 | 13.455 (12.641- | | 12.696 (11.703- | | <0.001 | 13.455 (12.578- | | 13.048 (11.703- | | <0.001 |
| | 14.379) | | 14.14) | | | 14.379) | | 13.763) | | |
| Index-OS | 22.964 (23.784- | | 21.667 (22.192- | | <0.001 | | | | | |
| | 19.309) | | 19.309) | | | | | | | |
| Index-PFS | | | | | | 21.342 (20.744- | | 20.178 (20.737- | | <0.001 |
| | | | | | | 22.134) | | 18.177) | | |
| Characteristic | | | Index-OS | | | | | | | |

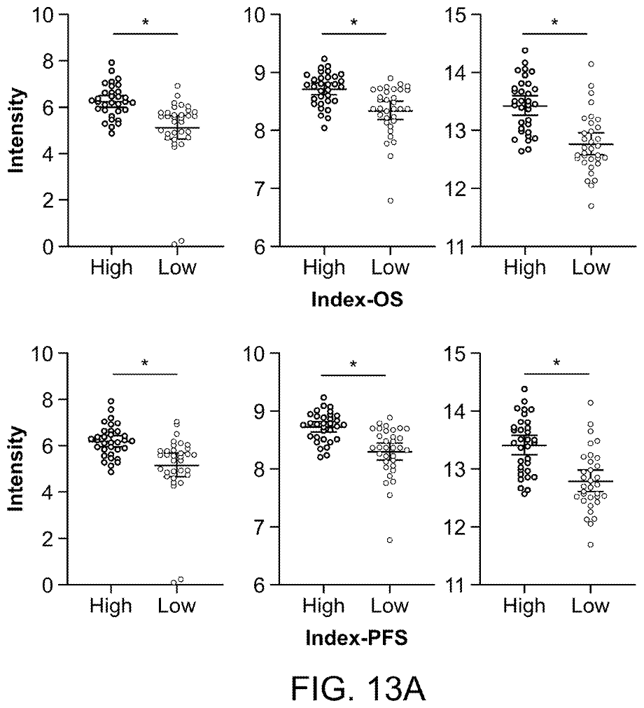

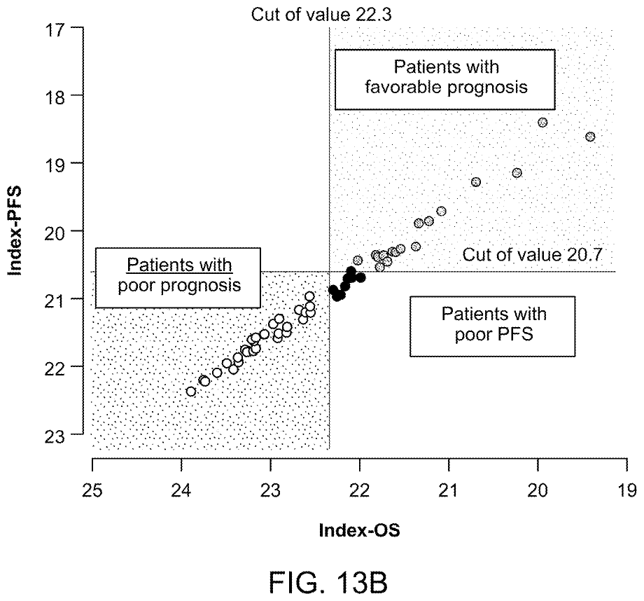

The expression of three miRNA was bifurcated according to the indices-OS and -PFS, as shown in FIG. 13A. Finally, the correlation between the indices-OS and -PFS was evaluated using the Pearson correlation coefficient, which revealed a significant correlation (R²=0.992, p<0.001), as shown in FIG. 13B.

### Example 4: Predicting Cancer Outcomes

Provided herein are methods to identify miRNAs as markers to predict a cancer outcome in a subject.

To predict the cancer outcome, a number of subjects are selected. The selected subjects may share a common cancer, a type of cancer, or a subtype of cancer. The selected subjects may not have other cancers, types of cancers, or subtypes of cancers. A sample is obtained each of the subjects. The sample comprises a cell-free or bodily fluid sample. The sample comprises a plurality of miRNAs. The levels of at least one of the miRNAs is provided to an algorithm, such as a statistical model. The algorithm generates an formula comprising the levels of a subset of the miRNAs provided. The formula will then be used to calculate a disease outcome index. The formula is then used to stratify the subjects into two populations based on a high or low index value, wherein the two populations have different disease outcomes or predicted disease outcome.

### EMBODIMENTS

1. A method, comprising:
(a) obtaining an index derived from at least two micro-ribonucleic acids (miRNAs) of a subject having an ovarian cancer;
(b) determining an outcome of said ovarian cancer in said subject, wherein said at least two miRNAs are obtained from a cell-free sample of said subject.
   2. The method of embodiment 1, wherein said ovarian cancer comprises a type I ovarian cancer, a type II ovarian cancer, or a combination thereof.
   3. The method of embodiment 2, wherein said ovarian cancer comprises said type I ovarian cancer.
   4. The method of embodiment 3, wherein said type I ovarian cancer comprises endometrioid carcinoma, ovarian clear cell carcinoma, mucinous carcinoma, low-grade serous carcinoma, or a combination thereof.
   5. The method of embodiment 4, wherein said type I ovarian cancer comprises said endometrioid carcinoma.
   6. The method of embodiment 4, wherein said type I ovarian cancer comprises said ovarian clear cell carcinoma.
   7. The method of embodiment 4, wherein said type I ovarian cancer comprises said mucinous carcinoma.
   8. The method of embodiment 4, wherein said type I ovarian cancer comprises said low-grade serous carcinoma.
   9. The method of any one of embodiments 2-8, wherein said ovarian cancer comprises said type II ovarian cancer.
   10. The method of embodiment 9, wherein said type II ovarian cancer comprises high-grade serous ovarian carcinoma.
   11. The method of any one of embodiments 1-10, wherein said ovarian cancer comprises epithelial ovarian carcinoma, germ cell tumor, stromal cell tumor, or a combination thereof.
   12. The method of embodiment 11, wherein said ovarian cancer comprises said epithelial ovarian carcinoma.
   13. The method of embodiment 11, wherein said ovarian cancer comprises said germ cell tumor.
   14. The method of embodiment 11, wherein said ovarian cancer comprises said stromal cell tumor.
   15. The method of any one of embodiments 1-14, wherein said at least two miRNAs comprise miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, miR-6805-5p, or a combination thereof.
   16. The method of any one of embodiments 1-15, wherein said at least two miRNAs comprise miR-187-5p, miR-6870-5p, miR-1908-5p, miR-6727-5p, or a combination thereof.
   17. The method of any one of embodiments 1-16, wherein said at least two miRNAs comprise miR-187-5p.
   18. The method of any one of embodiments 1-17, wherein said at least two miRNAs comprise miR-6870-5p.
   19. The method of any one of embodiments 1-18, wherein said at least two miRNAs comprise miR-1908-5p.
   20. The method of any one of embodiments 1-19, wherein said at least two miRNAs comprise miR-6727-5p.
   21. The method of any one of embodiments 1-20, wherein said at least two miRNAs comprise miR-187-5p and miR-6870-5p.
   22. The method of any one of embodiments 1-21, wherein said at least two miRNAs comprise miR-187-5p, miR-6870-5p, and miR-1908-5p.
   23. The method of any one of embodiments 1-22, wherein said at least two miRNAs comprise miR-187-5p, miR-6870-5p, and miR-6727-5p.
   24. The method of any one of embodiments 1-23, wherein said at least two miRNAs comprise miR-187-5p, miR-6870-5p, miR-1908-5p, and miR-6727-5p.
   25. The method of any one of embodiments 1-24, wherein said at least two miRNAs comprise miR-150-3p, miR-3195, miR-7704, or a combination thereof.
   26. The method of any one of embodiments 1-25, wherein said at least two miRNAs comprise miR-150-3p.
   27. The method of any one of embodiments 1-26, wherein said at least two miRNAs comprise miR-3195.
   28. The method of any one of embodiments 1-27, wherein said at least two miRNAs comprise miR-7704.
   29. The method of any one of embodiments 1-28, wherein said at least two miRNAs comprise miR-150-3p, miR-3195 and miR-7704.
   30. The method of any one of embodiments 1-29, wherein said index is derived from levels of said at least two miRNAs.
   31. The method of embodiment 30, wherein said levels of said at least two miRNAs comprise expression levels of said at least two miRNAs or derivatives of said expression levels of said at least two miRNAs.
   32. The method of embodiment 30 or 31, wherein said index is obtained by processing said levels of said at least two miRNAs of at least two subjects having said ovarian cancer.
   33. The method of any one of embodiments 30-32, wherein said index is obtained by processing said levels of said at least two miRNAs of at least 10 subjects having said ovarian cancer.
   34. The method of any one of embodiments 30-33, wherein said index is obtained by processing said levels of said at least at least two miRNAs of at least 50 subjects having said ovarian cancer.
   35. The method of any one of embodiments 30-34, wherein said index is obtained by processing said levels of said at least at least two miRNAs of at least 100 subjects having said ovarian cancer.
   36. The method of any one of embodiments 30-35, wherein said index comprises a formula comprising said levels of said at least two miRNAs.
   37. The method of embodiment 36, wherein said formula comprises:
(a) 0.218×(a level of miR-187-5p)+0.280×(a level of miR-6870-5p); or
(b) 0.148×(said level of miR-187-5p)+0.273×(said level of miR-6870-5p)+0.186×(a level of miR-1908-5p); or
(c) 0.034×(said level of miR-187-5p)+0.236×(said level of miR-6870-5p)+0.504×(a level of miR-6727-5p)+0.048×(said level of miR-1908-5p); or
(d) 0.031×(said level of miR-187-5p)+0.231 (said level of miR-6870-5p)+0.351×(said level of miR-6727-5p).

38. The method of embodiment 36, wherein said formula comprises:
(a) 0.463×(a level of miR-150-3p)+1.323×(a level of miR-3195)+0.636×(a level of miR-7704); or
(b) 0.399×(said level of miR-150-3p)+1.426×(said level of miR-3195)+0.480×(said level of miR-7704)
   39. The method of any one of embodiments 30-38, wherein said index is obtained by processing said levels of said at least two miRNAs using an algorithm.
   40. The method of embodiment 39, wherein said algorithm comprises a statistical model.
   41. The method of embodiment 40, wherein said statistical model comprises a linear regression model.
   42. The method of embodiment 41, wherein said linear regression model comprises a Cox model.
   43. The method of embodiment 42, wherein said Cox model comprises a univariate Cox model or a multivariate Cox model.
   44. The method of embodiment 43, wherein said Cox model comprises said univariate Cox model.
   45. The method of embodiment 43, wherein said Cox model comprises said multivariate Cox model.
   46. The method of any one of embodiments 30-45, wherein said levels of said at least two miRNAs are determined by a microarray, a sequencing reaction, a probe hybridization, a polymerase chain reaction (PCR), or a combination thereof.
   47. The method of embodiment 46, wherein said levels of said at least two miRNAs are determined by said microarray.
   48. The method of any one of embodiments 1-47, wherein said ovarian cancer is a stage I ovarian cancer, a stage II ovarian cancer, a stage III ovarian cancer, or a stage IV ovarian cancer.
   49. The method of embodiment 48, wherein said stage I ovarian cancer comprises a stage IA ovarian cancer or a stage IB ovarian cancer.
   50. The method of embodiment 48, wherein said stage II ovarian cancer comprises a stage IIA ovarian cancer or a stage IIB ovarian cancer.
   51. The method of embodiment 48, wherein said stage III ovarian cancer comprises a stage IIIA ovarian cancer, a stage IIIB ovarian cancer, or a stage IIIC ovarian cancer.
   52. The method of any one of embodiments 1-51, wherein said outcome comprises an amount of time that said ovarian cancer has not progressed to a succeeding stage, during or subsequent to when said subject has received a treatment for said ovarian cancer.
   53. The method of any one of embodiments 1-51, wherein said outcome comprises an amount of time that said subject is alive, subsequent to said subject has been determined to have said ovarian cancer or said subject has received a treatment for said ovarian cancer.
   54. The method of any one of embodiments 1-51, wherein said outcome comprises progress-free survival (PFS) or overall survival (OS).
   55. The method of embodiment 54, wherein said outcome comprises said PFS.
   56. The method of embodiment 54, wherein said comprises said OS.
   57. The method of any one of embodiments 1-56, wherein said cell-free sample of said subject comprises a bodily fluid of said subject.
   58. The method of embodiment 57, wherein said bodily fluid comprises serum, urine, sweat, plasma, tears, semen, vaginal fluid, amniotic fluid, milk, or a combination thereof.
   59. The method of embodiment 58, wherein said bodily fluid sample comprises said serum.
   60 The method of any one of embodiments 1-59, wherein said at least two miRNAs are derived from extracellular vesicles of said subject.

While the invention has been described with reference to the aforementioned specification, the descriptions and illustrations of the embodiments herein are not meant to be construed in a limiting sense. Numerous variations, changes, and substitutions may now occur to those skilled in the art without departing from the invention. Furthermore, it shall be understood that all aspects of the invention are not limited to the specific depictions, configurations or relative proportions set forth herein which depend upon a variety of conditions and variables. It may be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-60. (canceled)

61. A method, comprising:
(a) obtaining an index derived from at least two micro-ribonucleic acids (miRNAs) of a subject having an ovarian cancer;
(b) determining an outcome of said ovarian cancer in said subject using said index,
wherein said at least two miRNAs are obtained from a cell-free sample of said subject, and wherein said outcome comprises:
i. an amount of time that said ovarian cancer in said subject has not progressed to a succeeding cancer stage, (1) subsequent to when said subject is determined to have said ovarian cancer, (2) while said subject is receiving a treatment for said ovarian cancer, or (3) subsequent to when said subject receives said treatment for said ovarian cancer;
ii. an amount of time that said subject is alive, subsequent to (1) when said subject is determined to have said ovarian cancer or (2) when said subject receives said treatment for said ovarian cancer; or
iii. a combination of (i) and (ii).

62. The method of claim 61, wherein said outcome comprises said amount of time that said ovarian cancer in said subject has not progressed to a succeeding cancer stage, (1) subsequent to when said subject is determined to have said ovarian cancer, (2) while said subject is receiving said treatment for said ovarian cancer, or (3) subsequent to when said subject receives said treatment for said ovarian cancer.

63. The method of claim 61, wherein said outcome comprises said amount of time that said subject is alive, subsequent to (1) when said subject is determined to have said ovarian cancer or (2) when said subject receives said treatment for said ovarian cancer.

64. The method of claim 61, wherein said outcome further comprises progress-free survival (PFS), overall survival (OS), or a combination thereof.

65. The method of claim 61, wherein said ovarian cancer comprises a type I ovarian cancer, a type II ovarian cancer, or a combination thereof.

66. The method of claim 61, wherein said ovarian cancer comprises endometrioid carcinoma, ovarian clear cell carcinoma, mucinous carcinoma, low-grade serous carcinoma, high-grade serous ovarian carcinoma, epithelial ovarian carcinoma, germ cell tumor, stromal cell tumor, or a combination thereof.

67. The method of claim 61, wherein said at least two miRNAs comprise at least one of miR-187-5p, miR-6870-5p, miR-1908-5p, miR-6727-5p, miR-711, miR-1229-5p, miR-1914-3p, miR-4513, miR-4656, miR-4787-3p, miR-6787-5p, miR-6850-5p, miR-7107-5p, miR-7150, miR-150-3p, miR-3195, miR-7704, miR-365a-5p, miR-4730, miR-671-5p, or miR-6805-5p.

68. The method of claim 61, further comprising processing levels of said at least two miRNAs or derivatives thereof to generate said index.

69. The method of claim 68, further comprising processing said levels of said at least two miRNAs or said derivatives thereof of at least 2 subjects, at least 10 subjects, at least 50 subjects, or at least 100 subjects having said ovarian cancer to generate said index.

70. The method of claim 68, further comprising processing said levels of said at least two miRNAs or said derivatives thereof using an algorithm to generate said index.

71. The method of claim 70, wherein said algorithm comprises a linear regression model.

72. The method of claim 71, wherein said linear regression model comprises a Cox model.

73. The method of claim 68, wherein said index comprises a formula comprising said levels of said at least two miRNAs or said derivatives thereof, and wherein said formula comprises:
$$( a ) 0.218 \times ( a level of miR - 187 - 5 p ) + 0.28 \times ( a level of miR - 6870 - 5 p ) ;$$
$$( b ) 0.148 \times ( said level of miR - 187 - 5 p ) + 0.273 \times ( said level of miR - 6870 - 5 p ) + 0.186 \times ( a level of miR - 1908 - 5 p ) ;$$
$$( c ) 0.034 \times ( said level of miR - 187 - 5 p ) + 0.236 \times ( said level of miR - 6870 - 5 p ) + 0.504 \times ( a level of miR - 6727 - 5 p ) + 0.048 \times ( said level of miR - 1908 - 5 p ) ;$$
$$( d ) 0.031 \times ( said level of miR - 187 - 5 p ) + 0.231 ( said level of miR - 6870 - 5 p ) + 0.351 \times ( said level of miR - 6727 - 5 p ) ;$$
$$( e ) 0.463 \times ( a level of miR - 150 - 3 p ) + 1.323 \times ( a level of miR - 3195 ) + 0.636 \times ( a level of miR - 7704 ) ; or$$
$$( f ) 0.399 \times ( said level of miR - 150 - 3 p ) + 1.426 \times ( said level of miR - 3195 ) + 0.48 \times ( said level of miR - 7704 ) .$$

74. The method of claim 61, wherein said cell-free sample comprises a bodily fluid.

75. The method of claim 74, wherein said bodily fluid comprises serum, urine, sweat, plasma, tears, semen, vaginal fluid, amniotic fluid, milk, or a combination thereof.

76. The method of claim 75, wherein said bodily fluid sample comprises said serum.

77. The method of claim 61, wherein said at least two miRNAs are cell-free miRNAs.

78. The method of claim 77, wherein said at least two miRNAs are derived from extracellular vesicles of said subject.

79. The method of claim 61, further comprising extracting said at least two miRNAs from said subject.

80. The method of claim 61, wherein said treatment comprises surgical operations, chemotherapy, or radiotherapy.
